SEQUENCE LISTING (6 sequences)

SEQ ID NO: 1 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 2 - DNA, 852 bp, Homo sapiens
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatga

SEQ ID NO: 3 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 4 - DNA, 1059 bp, Homo sapiens
atggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccaccaccacgccgcccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtag

SEQ ID NO: 5 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 6 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatctcgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccgcacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

(19) United States
(12) Patent Application Publication
(10) Pub. No.: US 2020/0109370 A1
(43) Pub. Date: Apr. 9, 2020

(54) COMPOSITIONS AND METHODS FOR PROVIDING CELL REPLACEMENT THERAPY

(71) Applicants: ORGENESIS LTD., Tel Aviv (IL); TEL HASHOMER MEDICAL RESEARCH INFRASTRUCTURE AND SERVICES LTD., Ramat Gan (IL)

(72) Inventor: Sarah FERBER, Tel Aviv (IL)

(73) Assignees: ORGENESIS LTD., Tel Aviv (IL); TEL HASHOMER MEDICAL RESEARCH INFRASTRUCTURE AND SERVICES LTD., Ramat Gan (IL)

(21) Appl. No.: 16/618,119

(22) PCT Filed: May 27, 2018
(86) PCT No.: PCT/IL2018/050579

§ 371 Date: Nov. 28, 2019

Related U.S. Application Data
(60) Provisional application No. 62/512,083, filed on May 29, 2017.
(60) Provisional application No. 62/512,085, filed on May 29, 2017.

(51) Int. Cl. C12N 5/071; C12N 15/86; A61K 35/407

(52) U.S. Cl. CPC C12N 5/0679; C12N 15/86; C12N 2533/40; C12N 2750/14143; C12N 2513/00; A61K 35/407

(57) ABSTRACT

Disclosed is a three-dimensional (3D) cell cluster comprising transdifferentiated adult mammalian non-pancreatic beta cells having a mature pancreatic beta cell phenotype and function and a scaffold, wherein said transdifferentiated cells have an enhanced mature pancreatic beta cell phenotype compared to a 3D cell cluster without a scaffold and to similarly transdifferentiated cells cultured as a two-dimensional (2D) monolayer.

### FIELD OF DISCLOSURE

The disclosure presented herein provides three-dimensional (3D) cell clusters comprising transdifferentiated adult mammalian non-pancreatic beta cells having a mature pancreatic beta cell phenotype and a scaffold, and methods of generating thereof. Also disclosed herein are methods for treating a pancreatic disorder with said 3D clusters.

### BACKGROUND

Diabetes mellitus, commonly referred to as diabetes, is a clinical disorder characterized by the inadequate secretion and/or utilization of insulin resulting in a life-threatening condition that is projected to be the 7th leading cause of death in 2030. Treatment options for diabetes are centered on self-injection of insulin, which is an inconvenient and imprecise solution. Pancreas transplantation is also considered in patients with severe complications of the disease. Although pancreas transplantation is associated with insulin independence in >80% of patients, it is a complicated procedure with significant morbidity and mortality.

Though most of the efforts to develop cell-based therapies for the treatment of diabetes make use of pancreatic islets, an increased research effort has been recently directed at the differentiation of cells from various sources into insulin producing cells (IPC). Reprogramming of adult human liver cells toward IPC by ectopic expression of pancreatic transcription factors (pTF) has been suggested as an unlimited source of β-cell replenishment. Transdifferentiated liver cells were shown to produce, process, and secrete insulin in a glucose-regulated manner, ameliorating hyperglycemia by in vivo implantation in diabetic SCID mice. To achieve insulin secretion, liver cells are transduced with pTF to induce differentiation into glucose regulated insulin-producing cells.

Cells in general and pancreatic β-cells in particular exist in three-dimensional (3D) microenvironments with intricate cell-cell and cell-matrix interactions and complex transport dynamics for nutrients and cells. Standard two-dimensional (2D), or monolayer, cell cultures are inadequate representations of this environment. 3D cell clusters more closely resemble in vivo tissue in terms of cellular communication and the development of extracellular matrices. These matrices help the cells function similar to the way cells would function in living tissue. In general, 3D cell cultures also have greater stability and longer lifespans than cell cultures in 2D. This means that they are more suitable for long-term implantation and for long-term effects of the cells on the host.

3D cell clusters might be grown on scaffolds, which provide structural support for cell attachment and tissue development. Scaffolds usually resemble the extracellular environment of cells by providing attachment sites and in some cases associated factors. Many synthetic and bioresorbable polymeric biomaterials are used for cell scaffolds. Among them, poly (glycerol sebacate) (PGS) has gained increased popularity due to its numerous advantages.

It is clear that there remains a critical need for improved treatment for diabetes. The 3D cell clusters disclosed herein have several features that make them advantageous over treatments for diabetes known in the art, as well as over other insulin producing cells (IPC). These clusters may be used in transplantation therapies, obviating the need for numerous self-injections of insulin, now required for the treatment of diabetes.

### SUMMARY OF THE DISCLOSURE

In one aspect, disclosed herein is a three-dimensional (3D) cell cluster comprising transdifferentiated adult mammalian non-pancreatic beta cells having a mature pancreatic beta cell phenotype and function and a scaffold, wherein at least a subset of said cells are attached to said scaffold.

In a related aspect, the scaffold is selected from a group comprising: a solid scaffold, a hydrogel, an extracellular matrix, an extracellular matrix hydrogel, a protein hydrogel, a peptide hydrogel, a polymer hydrogel, a wood-based nanocellulose hydrogel, polyglycerol sebacate (PGS), or any combination thereof.

In another aspect, the PGS is crosslinked with an agent. In a related aspect, the agent is selected from a group comprising laminin, fibronectin, fibrin, collagen types VIII, and elastin, and any combination thereof. In a related aspect, the scaffold comprises a plurality of microparticles. In a related aspect, the scaffold comprises PGS, and wherein said microparticles range from about 1 μm to about 1 mm in diameter.

In another aspect, the microparticles range from about 50 μm to about 500 μm in diameter. In a related aspect, the 3D cell cluster is encapsulated by an encapsulation agent. In a related aspect, the encapsulation agent comprises a material selected from a group comprising: alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, and low molecular weight dextran sulphate (LMW-DS), or any derivatives thereof, and any combination thereof.

In another aspect, the scaffold encapsulates the transdifferentiated cells. In a related aspect, the transdifferentiated cells comprise improved glucose regulated C-peptide secretion, improved glucose regulated insulin secretion, increased insulin content, increased expression of GCG, increased expression of NKX6.1, or increased expression of PAX6, or any combination thereof, compared to transdifferentiated non-pancreatic beta insulin producing cells cultured as a monolayer cell culture.

In another aspect, the transdifferentiated cells secrete at least 20 μm/h*10⁶ cells of C-peptide in response to high glucose concentrations. In a related aspect, the cells are transdifferentiated by ectopically expressed transcription factors, and the ectopically expressed pancreatic transcription factors have increased expression compared to transdifferentiated non-pancreatic beta cells having a mature pancreatic beta cell phenotype similarly transdifferentiated by ectopically expressed pancreatic transcription factors and cultured as a monolayer cell culture.

In another aspect, the viability of the transdifferentiated mammalian non-pancreatic beta insulin producing cells is similar to that of transdifferentiated mammalian non-pancreatic beta insulin producing cells cultured as a monolayer cell culture. In a related aspect, the adult mammalian non-pancreatic beta cells are selected from the group comprising epithelial cells, endothelial cells, keratinocytes, fibroblasts, muscle cells, hepatocytes, liver cells, blood cells, stem or progenitor cells, liver stem cells, neural stem cells, mesenchymal stem cells, hematopoietic stem or progenitor cells, or any combination thereof.

In a related aspect, the stem or progenitor cells are obtained from a tissue selected from a group comprising: bone marrow, umbilical cord blood, peripheral blood, fetal liver, and adipose tissue, or any combination thereof.

In one aspect, disclosed herein is a pharmaceutical composition comprising a 3D cell cluster comprising transdifferentiated adult mammalian non-pancreatic beta cells having a mature pancreatic beta cell phenotype and function and a scaffold, wherein at least a subset of said cells are attached to said scaffold.

In one aspect, disclosed herein is a method of generating a 3D cell cluster comprising transdifferentiated mammalian non-pancreatic beta cells having a mature pancreatic beta cell phenotype and function and a scaffold, wherein at least a subset of said cells are attached to said scaffold, the method comprising: providing the scaffold; obtaining primary adult mammalian non-pancreatic cells; propagating and expanding the primary adult mammalian non-pancreatic cells of the previous step; transdifferentiating the propagated and expanded cells of the previous step; attaching at least a subset of the transdifferentiated cells of the previous step to the scaffold; thereby generating a 3D cell cluster comprising transdifferentiated mammalian non-pancreatic beta insulin producing cells, wherein at least a subset of said cells are attached to said scaffold.

In another aspect, the transdifferentiating comprises: infecting the expanded cells with an adenoviral vector comprising a nucleic acid encoding a human PDX-1 polypeptide, at a first timepoint; infecting the expanded cells of of the previous step with an adenoviral vector comprising a nucleic acid encoding a second human pancreatic transcription factor polypeptide at a second timepoint; and infecting the expanded cells of the previous step with an adenoviral vector comprising a nucleic acid encoding a human MafA polypeptide at a third timepoint.

In another aspect, the second pancreatic transcription factor is selected from NeuroD1 and Pax4. In a related aspect, the first timepoint and said second timepoint are concurrent. In a related aspect, the propagating and expanding of the cells, the transdifferentiating of the cells, or a combination thereof are executed under non-adherent cell culture conditions.

In one aspect, disclosed herein is a method for treating a pancreatic disease or disorder in a subject, the method comprising administering a 3D cell cluster comprising transdifferentiated mammalian non-pancreatic beta cells having a mature pancreatic beta cell phenotype and function and a scaffold to said subject; thereby treating the disease in the subject.

In another aspect, the administering comprises intradermal, intraperitoneal, or surgical administration, or any combination thereof, of the 3D cell cluster to said subject.

In a related aspect, the disease comprises type I diabetes, type II diabetes, gestational diabetes, pancreatic cancer, hyperglycemia, pancreatitis, pancreatic pseudocysts, pancreatic trauma caused by injury, type 3 diabetes or a complication of pancreatectomy, or any combination thereof.

### BRIEF DESCRIPTION OF THE DRAWINGS

FIG. 1 shows polyglycerol sebacate (PGS) and its synthesis. FIG. 1A shows a scanning electron microscope (SEM) image of 3D PGS microparticle aggregates. FIG. 1B shows the reaction scheme for the chemical synthesis of PGS.

FIG. 2 shows an overview of the three-dimensional (3D) cell cluster manufacturing process. Steps include: Optional Step 1—Obtaining liver tissue (e.g., a liver biopsy); Step 2—Processing of the tissue to recover primary liver cells; Step 3—Propagating the primary liver cells to predetermined cell number; Step 4—Transdifferentiation of the primary liver cells; Step 5—Culturing in non-Adherent Conditions; Step 6—Harvesting 3D Cell Clusters; and Step 7—Testing the transdifferentiated cells for quality assurance and quality control (i.e., safety, purity and potency). Optional steps include cryopreserving early passage liver cells; thawing cryopreserved cells for use at a later date; dissociating single cells from the 3D cluster; and storage of transdifferentiated cells for use at a later date.

FIGS. 3A-3D show an overview of the culture methods and protocols used in Examples 2-5. FIG. 3A shows a schematic draw of the methods used for culturing transdifferentiated (TD) liver cells in adherent (two-dimensional (2D)) and non-adherent (3D) conditions. FIG. 3B shows the different culture conditions studied. FIG. 3C shows the procedures performed on day 6 of the experiment. FIG. 3D shows the procedures performed on day 7 of the experiment.

FIG. 4 shows a schematic draw of the method used for transdifferentiating cells in non-adherent (3D) conditions for the experiments described in Examples 4 and 5.

FIG. 5 shows a schematic draw of the method used for culturing transdifferentiated (TD) liver cells in non-adherent conditions used in Example 6.

FIG. 6 shows 3D cell clusters of different sizes and the correlations between cluster size and the number of cells seeded.

FIGS. 7A-7C show the phenotype of TD cells seeded in different concentrations and grown in adherent (2D) and non-adherent (3D) conditions. FIG. 7A shows ectopic gene expression of PDX-1, NeuroD1 and MafA. FIG. 7B shows pancreatic gene expression of NKX6.1, somatostatin (SST) and glucagon (GCG). FIG. 7C shows C-peptide secretion.

FIG. 8 shows 3D cell clusters generated under non-adherent conditions in different media, and afterwards seeded in adherent conditions.

FIGS. 9A-9B show C-peptide secretion of transdifferentiated cells grown in adherent (2D) and non-adherent (3D) conditions in different media. FIG. 9A shows pmole of C-peptide secreted. FIG. 9B shows pmole of C-peptide per hour normalized according to μg RNA.

FIGS. 10A-10D show gene expression of TD cells grown in adherent (2D) and non-adherent (3D) conditions and in different media on Day 6 and Day 7. FIG. 10A shows ectopic expression of PDX-1, NeuroD1 and MafA. FIG. 10B shows expression of NKX6.1. FIG. 10C shows expression of GCG. FIG. 10D shows expression of PAX6.

FIGS. 11A-11C show the morphology of 3D cell clusters cultured under different conditions. FIG. 11A shows morphology of clusters generated by 2.5×10⁶ cells seeded in 75T flasks in 12.5 ml medium. FIG. 11B shows morphology clusters generated by 3×10⁵ cells seeded in 6 well plates in 4 ml medium. FIG. 11C shows morphology clusters generated by 3.75×10⁵ seeded in 6 well plates in 4 ml medium.

FIGS. 12A-12B show gene expression of TD cells grown in adherent (2D) and non-adherent (3D) conditions and in flasks of different sizes. FIG. 12A shows ectopic expression of PDX-1, NeuroD1 and MafA. FIG. 12B shows expression of the pancreatic-specific glucagon (GCG) and NKX6.1.

FIGS. 13A-13B show the phenotype of TD cells grown in adherent (2D) and non-adherent (3D) conditions and transdifferentiated with viruses manufactured by Pall Inc (USA). FIG. 13A shows gene expression of NKX6.1, glucagon (GCG) and somatostatin (SST). FIG. 13B shows ectopic expression of PDX-1, NeuroD1 and MafA.

FIG. 14 shows gene expression of TD cells grown in adherent (2D) and non-adherent (3D) conditions and transdifferentiated with viruses provided by different manufacturers. FIG. 14A shows gene expression of NKX6.1, glucagon (GCG) and somatostatin (SST). FIG. 14B shows ectopic expression of PDX-1, NeuroD1 and MafA. *: cells infected with OD260 Inc. (ID, USA) adenoviruses; **: cells infected with Pall Inc. (USA) adenoviruses.

FIGS. 15A-15B show C-peptide secretion of TD cells grown in adherent (2D) and non-adherent (3D) conditions and transdifferentiated with viruses provided by different manufacturers. FIG. 15A shows pmole/ml of C-peptide secreted. FIG. 15B shows pmole per hour per 10⁶ cells. *: cells infected with OD260 Inc. (ID, USA) adenoviruses; **: cells infected with Pall Inc. (USA) adenoviruses.

FIG. 16 shows representative clusters of untreated (UT) and transdifferentiated (TD) primary human adult liver cells cells grown in AggreWells (150 cells/well). Light microscopy images were taken on days 7 and 15. Upper panels show the aggregates that formed with 150 cells/well of UT and TD cells on

FIGS. 17A-17B show gene expression of transdifferentiated (TD) cells grown in adherent (2D) and non-adherent (3D) conditions. FIG. 17A shows ectopic expression of PDX-1, NeuroD1 and MafA. FIG. 17B shows endogenous gene expression of NKX6.1, and glucagon (GCG).

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers.

The disclosure relates to compositions and methods for providing transdifferentiated cells in scaffolds to treat pancreatic, liver, and other diseases. Further disclosed herein are three-dimensional (3D) cell clusters comprising transdifferentiated mammalian non-pancreatic beta insulin producing cells and a scaffold, wherein said clusters are attached to a scaffold. In some embodiments, transdifferentiated cells are capable of producing and secreting pancreatic hormones. In some embodiments, said cells are encapsulated within said scaffold. Further disclosed herein are methods for producing 3D cell clusters of transdifferentiated cells attached to scaffolds. Further disclosed herein are methods for treating a pancreatic disorder, the method comprising administering a 3D cell cluster of transdifferentiated cells attached to a scaffold to a subject in need thereof.

Three-dimensional (3D) Cell Clusters

In some embodiments, disclosed herein is a three-dimensional (3D) cell cluster comprising transdifferentiated mammalian non-pancreatic beta insulin producing cells (IPCs) and a scaffold, wherein at least a subset of said cells are attached to a scaffold. A skilled artisan would appreciate that the term “3D cell cluster” may encompass a group of cells physically contacting each other and organized in a three dimensional “3D” structure. A cell in a 3D cluster can contact other cells located in any direction relative to itself (i.e., above, below and on the laterals). A 3D cluster may be suspended in a culture medium, having all its external surface contacting the medium. This contrasts with two-dimensional “2D” cell clusters or other types of monolayer cell cultures. A cell in a 2D cluster is attached to the plate on one of its sides, and can only contact other cells located on its laterals. Similarly, only one side of 2D cluster can be in physical contact with the medium.

The term “3D cell cluster” may be used interchangeably with “cell spheroid”, “multicell spheroid”, “3D cell colonies”, and “cell cluster”, having all the same qualities and meanings.

In some embodiments, a 3D cell cluster has a size between about 10 to 50 μm. In some embodiments, a 3D cell cluster has a size between about 50 to 100 μm. In some embodiments, a 3D cell cluster has a size between about 100 to 200 μm. In some embodiments, a 3D cell cluster has a size between about 200 to 300 μm. In some embodiments, a 3D cell cluster has a size between about 300 400 μm. In some embodiments, a 3D cell cluster has a size between about 400 500 μm. In some embodiments, a 3D cell cluster has a size between about 500 to 600 μm. In some embodiments, a 3D cell cluster has a size between about 600 to 700 μm. In some embodiments, a 3D cell cluster has a size between about 700 to 800 μm. In some embodiments, a 3D cell cluster has a size between about 800 to 900 μm. In some embodiments, a 3D cell cluster has a size between about 900 to 1000 μm. In some embodiments, a 3D cell cluster has a size larger than 1000 μm.

In some embodiments, a 3D cell cluster comprises less than 10 cells. In some embodiments, a 3D cell cluster comprises between about 10 and 50 cells. In some embodiments, a 3D cell cluster comprises between about 50 and 500 cells. In some embodiments, a 3D cell cluster comprises between about 500 and 1000 cells. In some embodiments, a 3D cell cluster comprises between about 1000 and 2000 cells. In some embodiments, a 3D cell cluster comprises between about 2000 and 3000 cells. In some embodiments, a 3D cell cluster comprises between about 3000 and 4000 cells. In some embodiments, a 3D cell cluster comprises between about 4000 and 5000 cells. In some embodiments, a 3D cell cluster comprises more than 5000 cells.

In some embodiments, a 3D cell cluster comprises homogeneous cells. In some embodiments, a 3D cell cluster comprises heterogeneous cells. In some embodiments, a 3D cell cluster comprises cells comprising a similar phenotype. In some embodiments, a 3D cell cluster comprises cells comprising different phenotypes.

Scaffolds

In some embodiments, a subset of the transdifferentiated mammalian non-pancreatic beta insulin producing cells (IPC) are attached to a scaffold. In some embodiments, a subset of the cells comprises less than 10% of the cells. In some embodiments, a subset of the cells comprises between about 10% to 20% of the cells. In some embodiments, a subset of the cells comprises between about 20% to 30% of the cells. In some embodiments, a subset of the cells comprises between about 30% to 40% of the cells. In some embodiments, a subset of the cells comprises between about 40% to 50% of the cells. In some embodiments, a subset of the cells comprises between about 50% to 60% of the cells. In some embodiments, a subset of the cells comprises between about 60% to 70% of the cells. In some embodiments, a subset of the cells comprises between about 70% to 80% of the cells. In some embodiments, a subset of the cells comprises between about 80% to 90% of the cells. In some embodiments, a subset of the cells comprises between about 90% to 100% of the cells.

A skilled artisan would appreciate that the term “scaffold” encompasses an object providing structural support for cell attachment. Scaffolds are well known in the art and described, for example, in U.S. Pat. Nos. 6,379,962 and 6,143,293, which are each incorporated in their entirety herein by reference.

In some embodiments, the scaffold mimics the natural extracellular environment of the islets. In some embodiments, the scaffold provides resistance to hydrolytic or enzymatic degradation. In some embodiments, the scaffold mimics the hierarchical structure of the human pancreatic islets. In some embodiments, the scaffold encapsulates the cells in immune-protective biomaterials thus enhancing the transplant integration in the host. In some embodiments, scaffold porosity is tuned to promote oxygen and nutrient exchange, while preventing the entry of inflammatory cells and antibodies.

A skilled artisan would appreciate that the term “cell attachment” comprises the physical interaction of a cell to a surface, substrate or another cell, mediated by interaction of molecules of the cell surface, as cell adhesion molecules, selectins, integrins, syndecans, and cadherins. The term “cell attachment” may be used interchangeably with “cell adhesion”, “cell binding”, “cell loading”, and “cell association” having all the same qualities and meanings. In some embodiments, seeding a cell on a surface comprises attaching the cell to that surface. In some embodiments, cell attachment to a scaffold comprises non-covalent forces. In some embodiments, cells are covalently attached to a scaffold.

A skilled artisan would appreciate that the physico-mechanical, biochemical and functional characteristics of a scaffold can be assessed and optimized. The relevant physico-mechanical properties of the scaffold (e.g. elasticity, compressibility, viscoelastic behavior, tensile strength) can be studied, such as the mechanical properties which are influencing the cell adhesion and proliferation. The stability of the scaffolds under physiological conditions can be also assessed. For this purpose, the degradation of the scaffolds can be studied by exposing them to a combination of factors mimicking their natural environment in the site of transplantation (pH, enzymes, temperature, etc.). In vitro cell culture experiments can be performed to evaluate biocompatibility, cell attachment, cell viability and cell proliferation. Experiments can be performed to evaluate cell morphology by using contrast microscopy, cell recovery, and cell viability by using Trypan blue exclusion assay. Experiments can be performed to evaluate cell functionality at the molecular level, including assessing expression of pTF and hormones by real time PCR. Experiments can be performed to evaluate cell functionality at the cellular level, including assessing insulin content by dithizone staining, insulin secretion and content by assessment of C-peptide level by ELISA, and Glucose Stimulated Insulin Secretion (GSIS).

A skilled artisan would appreciate that the immunological profile of a scaffold can be assessed and optimized. Immunogenicity can be tested, for example by exposing peripheral blood mononuclear cells (PBMC) to the scaffold with or without transdifferentiated cells and measuring cytokines and T cell proliferation. Release of cytokines, as IFNγ, can be assessed by collecting PBMC supernatants following 48 hours and measuring cytokines by using commercially available kits. Proliferation of T cells can be assessed by Carboxyfluorescein succinimidyl ester (CFSE) staining following five days of co-incubation. CFSE labeling is diluted with each cell division and therefore it can be used to evaluate proliferations of T cells with flow cytometry. T cell subsets (CD8, CD4, T cells) can be labeled prior to the analysis. In vivo results can be validated by transplanting animals with the scaffold loaded with transdifferentiated cells or with the scaffold alone. In these in vivo experiments, mice are sacrificed at indicated time points post-transplantation and at each time point the transplant is retrieved. Half of the retrieved transplants are cultured, stained and observed under light and fluorescence microscopes to evaluate cell morphology, viability and tissue overgrowth. The other half of the retrieved microcapsules are used for histological analyses for identify reactive CD8 T cells.

A skilled artisan would appreciate that the effects of cell storage, package and transport on viability and function of transdifferentiated insulin producing cells attached to scaffolds can be assessed and optimized. Current methods for islets preservation are based on cold storage at 4° C. and allow for a limited viability of the cells of only 24-48 hours. The functionality of scaffold transdifferentiated cells can be tested at different temperatures and preservation media. Cell viability, gene expression and cell potency at several time points with or without the scaffold can be measured. Functional activity and potency at the end of the stability phase can be considered successful if they do not fall under 70% of the values achieved with the control product. Cells from at least three different donors can be tested. Two formulation solution candidates of transporting media can be used for comparison on the batches generated. The effect of Packaging material (mainly bags) can be established in terms of time, temperature, final cell density, and optimal application volume.

In some embodiments, the scaffold is a solid scaffold. In some embodiments, the scaffold comprises a hydrogel. In some embodiments, the scaffold comprises an extracellular matrix. In some embodiments, the scaffold comprises an extracellular matrix hydrogel. In some embodiments, the scaffold comprises a protein hydrogel. In some embodiments, the scaffold comprises a peptide hydrogel. In some embodiments, the scaffold comprises a polymer hydrogel. In some embodiments, the scaffold comprises a wood-based nanocellulose hydrogel. In some embodiments, the scaffold comprises polyglycerol sebacate (PGS). In some embodiments the scaffold is flexible and amenable to be fixed in place preventing its migration to an unintended location. In some embodiments, the scaffold encapsulates the cells. In some embodiments, the scaffold with the cells are encapsulated in an encapsulation agent.

In some embodiments, the cells attached to a scaffold are cells of the same type. In some embodiments, more than one type of cells is attached to a scaffold. In some embodiments, two types of cells are attached to a scaffold. In some embodiments, three types of cells are attached to a scaffold. In some embodiments, four types of cells are attached to a scaffold. In some embodiments, more than four types of cells are attached to a scaffold. In some embodiments, the cells attached to a scaffold are transdifferentiated insulin producing cells. In some embodiments, the cells attached to a scaffold are insulin producing cells and lymphocytes. In some embodiments, the cells attached to a scaffold are insulin producing cells and peripheral mononuclear blood cells (PBMC). In some embodiments, the cells attached to a scaffold are insulin producing cells, lymphocytes, and PBMC. In some embodiments, a type of cell attached to the scaffold provides supportive functions to transdifferentiated insulin producing cells. In some embodiments, a type of cells attached to the scaffold generates an immunotolerant environment. In some embodiments, an immunotolerant environment facilitates grafting and survival of the transplanted cells.

A skilled artisan would appreciate that the term “cell type” or “type of cell” comprises a classification used to distinguish between morphologically or phenotypically distinct cell forms. Some non-limiting examples of cell types comprise: epithelial cells, endothelial cells, keratinocytes, fibroblasts, muscle cells, hepatocytes, liver cells, blood cells, stem or progenitor cells, embryonic heart muscle cells, liver stem cells, neural stem cells, mesenchymal stem cells, hematopoietic stem and progenitor cells, insulin producing cells, transdifferentiated insulin producing cells, transdifferentiated cells having a pancreatic beta cell phenotype, transdifferentiated liver cells having a pancreatic beta cell phenotype, lymphocytes, PBMC, pancreatic cells other than pancreatic beta cells, acinar cells, alpha-cells.

A skilled artisan would appreciate that whereas virus transfection of liver cells is highly efficient in suspension, the transfection of adherent liver cells is challenging. Therefore, different scaffold architectures with different physico-mechanical and functional characteristics can be tested for their ability to promote transdifferentiation of liver cells while attached to the scaffolds. In a one embodiment, efficacy can be increased also by optimizing critical parameters as temperature and trypsin levels, as by using low concentration of EDTA.

In some embodiments, the scaffold improves the cell viability and function after storage, package and transport. In one embodiment, cell viability is increased for over 24 hours. In one embodiment, cell viability is increased for over 48 hours. In one embodiment, functional activity and potency at the end of the stability phase do not fall under 70% of the values achieved with the control product. A skilled artisan would appreciate that cell functionality can be also affected by modifying preservation temperature and preservation media. For this end, cell viability, gene expression and cell potency at several time points with or without the PGS scaffolding will be measured.

Polyglycerol Sebacate (PGS)

In one embodiment, the scaffold comprises polyglycerol sebacate (PGS). PGS is a polymer well known in the art and fully described, for example, in U.S. Pat. Nos. 9,359,472 and 7,722,894, which are incorporated by reference herein in their entirety. Methods for preparing PGS polymer are also well known in the art and fully described, for example, in U.S. Pat. Nos. 9,359,472 and 7,722,894.

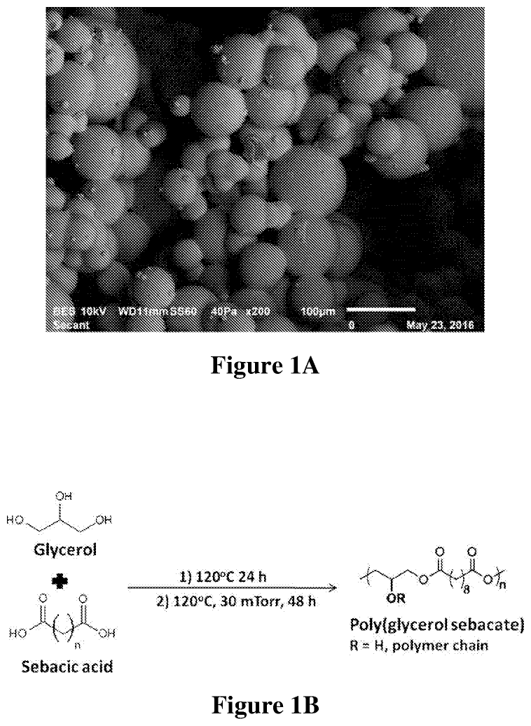

In some embodiments, a PGS polymer comprises a plurality of microparticles. Microparticles provide a high surface area to volume ratio for increased cell culture capacity (FIG. 1A). The common starting materials chosen for PGS synthesis are glycerol and sebacic acid (FIG. 1B). Glycerol (CH2(OH)CH(OH)CH2OH) is a basic building block for lipids which satisfies the design criteria mentioned above. Similarly, sebacic acid (HOOC(CH2)8COOH) is chosen as the acid monomer from the toxicological and polymer chemistry standpoints. PGS microparticles suitable for cell culture can be manufactured through any technique known in the art, for example, an emulsion technique combined with thermal curing. In one example, by changing temperature, shear mixing, the emulsion outer phase, and surfactant concentrations, the PGS microparticle size can be tuned. A skilled artisan would appreciate that different approaches can be used to increase the biocompatibility and alter the surface properties of PGS scaffolds for improved cell adhesion, expansion, and/or transdifferentiation. Hydrophilicity, surface charge, surface topography, and adsorbed biomolecules all influence cell attachment and can be modified through chemical etching techniques (e.g., NaOH treatment), enzyme treatment, grafting of hydrophilic groups, and coating the scaffold surface with adhesive proteins. Similarly, by modifying curing temperature, molar ratio of glycerol to sebacic acid, and curing time the degradation kinetics of PGS can be improved, thus decreasing PGS cytotoxic effects. A skilled artisan would appreciate that conditions that promote efficient attachment and even distribution of cells across the scaffold surface area can be optimized, for example, by altering scaffold concentrations, seeding densities, media formulations, minimal stir speeds, and medium components. High protein concentrations in medium, for example, can block or impede cell attachment to scaffolds. A skilled artisan would appreciate that experiments can be carried to determine the parameters needed for maintaining good oxygen transfer without creating high shear forces that separate the cells from the scaffolds. In these experiments, hydrodynamic shear stress is decreased as much as possible while monitoring that sufficient mixing is provided to maintain homogeneous conditions inside the bioreactor, to rapidly distribute feeds such as base or antifoam agent, and to maintain adequate absorption of oxygen and desorption of carbon dioxide in the cultivation medium for the respiration of the cells. Said experiments can be carried out, for example, in Wave bioreactors equipped with 5L single use bags.

In some embodiments, the PGS scaffold is crosslinked with an agent. In some embodiments, said agent comprises laminin In some embodiments, said agent comprises fibronectin. In some embodiments, said agent comprises fibrin. In some embodiments, said agent comprises collagen types I/III. In some embodiments, said agent comprises elastin. In some embodiments, the PGS scaffold is crosslinked with a combination of agents.

In one embodiment, the scaffold comprises a plurality of microparticles. In one embodiment, the microparticles range from about 0.1 μm to about 1 μm in diameter. In one embodiment, the microparticles range from about 1 μm to about 10 μm in diameter. In one embodiment, the microparticles range from about 10 μm to about 100 μm in diameter. In one embodiment, the microparticles range from about 50 μm to about 500 μm in diameter. In one embodiment, the microparticles range from about 100 μm to about 1 mm in diameter. In one embodiment, the microparticles range from about 1 mm to about 10 mm in diameter. In one embodiment, the microparticles range from about 1 μm to about 1 mm in diameter.

Encapsulation

In some embodiments, a 3D cell cluster comprising transdifferentiated mammalian non-pancreatic beta IPC and a scaffold, wherein at least a subset of said cells are attached to said scaffold, is encapsulated by an encapsulation agent. A skilled artisan would appreciate that the term “encapsulation agent” refers to a polymeric semi-permeable membrane that surrounds the cells and selectively permits the bidirectional diffusion of desired molecules, including the influx of molecules essential for cell metabolism and the efflux of molecules of therapeutic value and waste products. In some embodiments, the encapsulation agent protects transdifferentiated cells from immune rejection by the patient. In some embodiments, the encapsulation agent increases transdifferentiated cells viability compared to non-encapsulated transdifferentiated cells. In some embodiments, the encapsulation agent increases insulin secretion from transdifferentiated cells compared to non-encapsulated transdifferentiated cells. A skilled artisan would appreciate that the term “encapsulate” refers to enclosing an object within a membrane. In some embodiments, the membrane comprises a polymer semi-permeable membrane.

In some embodiments, mammalian non-pancreatic beta cells are encapsulated and then attached to a scaffold. In some embodiments, at least part of the mammalian non-pancreatic beta cells are encapsulated within the scaffold. In some embodiments, most mammalian non-pancreatic beta cells are encapsulated within said scaffold. In some embodiments, all mammalian non-pancreatic beta cells are encapsulated within said scaffold. In some embodiments, non-pancreatic beta cells are seeded on a scaffold, and subsequently the scaffold with the cells are encapsulated in an encapsulation agent. In some embodiments, soluble factors are included within the encapsulation agent. In some embodiments, factors promoting cell transdifferentiation are included within the encapsulation agent. In some embodiments, factors promoting cell survival are included within the encapsulation agent. A skilled artisan would appreciate that implantation of transdifferentiated cells encapsulated within semi-permeable membranes presents a number of advantages. First, encapsulated grafts are not rejected by the immune system. Second, encapsulation increases graft survival. Third, encapsulation reduces undesirable side-effects. Fourth, encapsulation reduces the need for long-term use of immunosuppressive drugs. Additionally, encapsulation allows grafts to be retrieved from the patient, in order to follow up cell potency in vivo, or in case the graft is damaging or risking the implanted patient.

In some embodiments, an encapsulating agent comprises alginate. In some embodiments, an encapsulating agent comprises cellulose sulphate. In some embodiments, an encapsulating agent comprises collagen. In some embodiments, an encapsulating agent comprises chitosan. In some embodiments, an encapsulating agent comprises gelatin. In some embodiments, an encapsulating agent comprises agarose. In some embodiments, an encapsulating agent comprises polyethylene glycol (PEG). In some embodiments, an encapsulating agent comprises poly-L-lysine (PLL). In some embodiments, an encapsulating agent comprises polysulphone (PSU). In some embodiments, an encapsulating agent comprises polyvinyl alcohol (PVA). In some embodiments, an encapsulating agent comprises polylactic acid (PLA). In some embodiments, an encapsulating agent comprises acrylates. In some embodiments, an encapsulating agent comprises low molecular weight dextran sulphate (LMW-DS). In some embodiments, an encapsulating agent comprises a derivative of the above disclosed materials. In some embodiments, an encapsulating agent comprises any combination of the above disclosed materials.

Transdifferentiated Cells

A skilled artisan would appreciate that the term “transdifferentiation” may encompass the process by which a first cell type loses identifying characteristics and changes its phenotype to that of a second cell type without going through a stage in which the cells have embryonic characteristics. In some embodiments, the first and second cells are from different tissues or cell lineages. In some embodiments, transdifferentiation involves converting a mature or differentiated cell to a different mature or differentiated cell. Any means known in the art for differentiating or transdifferentiating cells can be utilized. Specifically, lineage-specific transcription factors (TF) have been suggested to display instructive roles in converting adult cells to endocrine pancreatic cells, neurons, hematopoietic cells and cardiomyocyte lineages, suggesting that transdifferentiation processes occur in a wide spectrum of milieus. In all transdifferentiation protocols, ectopic transcription factors serve as a short-term trigger to a potential wide, functional and irreversible developmental process.

In some embodiments, transdifferentiation comprises the differentiation of progenitor cells of pancreatic beta cell lineage, such as pluripotent stem cells, endodermal cells, pancreatic stem cells, pancreatic stem cells, endocrine progenitor cells, or progenitors of the endocrine islet lineage.

A skilled artisan would appreciate that, in some embodiments, a mature pancreatic beta cell phenotype comprises the ability of the cells to engage in at least one of the following actions: glucose-sensing (for which the expression of GLUT2 (in mice) and GLUT1 (in humans) is needed), cell excitability (for which the expression of SUR1 and KIR6.2 is needed), insulin processing (for which the expression of PCSK1 and PCSK2 is needed), uptake of zinc into insulin-secretory granules (for which the expression of ZNT8 is needed), and secretion of chromogranin-B (CHGB) and urocortin 3 (UCN3). In some embodiments a mature pancreatic beta cell phenotype comprises the expression of UCN3, ZNT8, MAFA, CX36, PSCK1, PSCK2, MafB (in humans), PAX4, NEUROD1, ISL1, NKX6.1, GLUT2, INS, and PDX-1. In some embodiments, a mature pancreatic beta cell phenotype comprises the inactivation of the genes MAFB (in mice) and NGN3.

In some embodiment, a mature pancreatic beta cell phenotype and function comprises expression, production, and/or secretion of pancreatic hormones. Pancreatic hormones can comprise, but are not limited to, insulin, somatostatin, glucagon (GCG), or islet amyloid polypeptide (IAPP). Insulin can be hepatic insulin or serum insulin In some embodiments, the insulin is a fully process form of insulin capable of promoting glucose utilization, and carbohydrate, fat and protein metabolism. In some embodiments, a mature pancreatic beta cell phenotype and function comprises expression and/or production of pancreatic transcription factors. Pancreatic transcription factors can comprise Pdx1, Ngn3, NeuroD1, Pax4, MafA, NKX6.1, NKX2.2, Hnf1α, Hnf4α, Foxo1, CREB family members, NFAT, FoxM1, Snail and/or Asc-2.

In some embodiments, the pancreatic hormone is in a “prohormone” form. In other embodiments, the pancreatic hormone is in a fully processed biologically active form of the hormone. In other embodiments, the pancreatic hormone is under regulatory control i.e., secretion of the hormone is under nutritional and hormonal control similar to endogenously produced pancreatic hormones. For example, in some embodiments disclosed herein, the hormone is under the regulatory control of glucose.

The pancreatic beta cell phenotype can be determined for example by measuring pancreatic hormone production, i.e., insulin, somatostatin or glucagon protein mRNA or protein expression. Hormone production can be determined by methods known in the art, i.e. immunoassay, Western blot, receptor binding assays or functionally by the ability to ameliorate hyperglycemia upon implantation in a diabetic host. Insulin secretion can also be measured by, for example, C-peptide processing and secretion. In another embodiment, high-sensitivity assays may be utilized to measure insulin secretion. In another embodiment, high-sensitivity assays comprise an enzyme-linked immunosorbent assay (ELISA), a mesoscale discovery assay (MSD), or an Enzyme-Linked ImmunoSpot assay (ELISpot), or an assay known in the art.

In some embodiments, the cells may be directed to produce and secrete insulin using the methods specified herein. The ability of a cell to produce insulin can be assayed by a variety of methods known to those of ordinary skill in the art. For example, insulin mRNA can be detected by RT-PCR or insulin may be detected by antibodies raised against insulin. In addition, other indicators of pancreatic differentiation include the expression of the genes Isl-1, Pdx-1, Pax-4, Pax-6, and Glut-2. Other phenotypic markers for the identification of islet cells are disclosed in U.S. 2003/0138948, incorporated herein in its entirety.

The pancreatic beta cell phenotype can be determined for example by promoter activation of pancreas-specific genes. Pancreas-specific promoters of particular interest include the promoters for insulin and pancreatic transcription factors, i.e. endogenous PDX-1. Promoter activation can be determined by methods known in the art, for example by luciferase assay, EMSA, or detection of downstream gene expression.

In some embodiments, the pancreatic beta-cell phenotype can also be determined by induction of a pancreatic gene expression profile. A skilled artisan would appreciate that the term “pancreatic gene expression profile” may encompass a profile to include expression of one or more genes that are normally transcriptionally silent in non-endocrine tissues, i.e., a pancreatic transcription factor, pancreatic enzymes or pancreatic hormones. Pancreatic enzymes are, for example, PCSK2 (PC2 or prohormone convertase), PC1/3 (prohormone convertase 1/3), glucokinase, glucose transporter 2 (GLUT-2). Pancreatic-specific transcription factors include, for example, Nkx2.2, Nkx6.1, Pax-4, Pax-6, MafA, NeuroD1, NeuroG3, Ngn3, beta-2, ARX, BRAIN4 and Isl-1.

Induction of the pancreatic gene expression profile can be detected using techniques well known to one of ordinary skill in the art. For example, pancreatic hormone RNA sequences can be detected in, e.g., Northern blot hybridization analyses, amplification-based detection methods such as reverse-transcription based polymerase chain reaction or systemic detection by microarray chip analysis. Alternatively, expression can be also measured at the protein level, i.e., by measuring the levels of polypeptides encoded by the gene. In a specific embodiment PC1/3 gene or protein expression can be determined by its activity in processing prohormones to their active mature form. Such methods are well known in the art and include, e.g., immunoassays based on antibodies to proteins encoded by the genes, or HPLC of the processed prohormones.

In some embodiments, transdifferentiated non-pancreatic beta insulin producing cells cultured as a 3D cluster with a scaffold comprise increased glucose regulated C-peptide secretion compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased glucose regulated C-peptide secretion compared to transdifferentiated non-pancreatic beta insulin producing cells cultured as a 3D cell cluster without a scaffold.

In some embodiments, said increase in glucose regulated C-peptide secretion is less than 10%. In some embodiments, said increase in glucose regulated C-peptide secretion is between about 10% to 100%. In some embodiments, said increase in glucose regulated C-peptide secretion is between about 200% to 300%. In some embodiments, said increase in glucose regulated C-peptide secretion is between about 300% to 400%. In some embodiments, said increase in glucose regulated C-peptide secretion is between about 400% to 500%. In some embodiments, said increase in glucose regulated C-peptide secretion is between about 500% to 600%. In some embodiments, said increase in glucose regulated C-peptide secretion is between about 600% to 700%. In some embodiments, said increase in glucose regulated C-peptide secretion is between about 700% to 800%. In some embodiments, said increase in glucose regulated C-peptide secretion is between about 800% to 900%. In some embodiments, said increase in glucose regulated C-peptide secretion is between about 900% to 1000%. In some embodiments, said increase in glucose regulated C-peptide secretion is greater than 1000%.

In some embodiments, transdifferentiated non-pancreatic beta insulin producing cells cultured as a 3D cluster with a scaffold comprise increased glucose regulated insulin secretion compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased glucose regulated insulin secretion compared to transdifferentiated non-pancreatic beta insulin producing cells cultured as a 3D cell cluster without a scaffold.

In some embodiments, said increase in glucose regulated insulin secretion is less than 10%. In some embodiments, said increase in glucose regulated insulin secretion is between about 10% to 100%. In some embodiments, said increase in glucose regulated insulin secretion is between about 200% to 300%. In some embodiments, said increase in glucose regulated insulin secretion is between about 300% to 400%. In some embodiments, said increase in glucose regulated insulin secretion is between about 400% to 500%. In some embodiments, said increase in glucose regulated insulin secretion is between about 500% to 600%. In some embodiments, said increase in glucose regulated insulin secretion is between about 600% to 700%. In some embodiments, said increase in glucose regulated insulin secretion is between about 700% to 800%. In some embodiments, said increase in glucose regulated insulin secretion is between about 800% to 900%. In some embodiments, said increase in glucose regulated insulin secretion is between about 900% to 1000%. In some embodiments, said increase in glucose regulated insulin secretion is between above 1000%.

In some embodiments, transdifferentiated non-pancreatic beta insulin producing cells cultured as a 3D culture with a scaffold comprise increased insulin content compared to transdifferentiated non-pancreatic beta insulin producing cells cultured as a monolayer cell culture. In some embodiments, transdifferentiated non-pancreatic beta insulin producing cells cultured as a 3D culture with a scaffold comprise increased insulin content compared to transdifferentiated non-pancreatic beta insulin producing cells cultured as a 3D cell cluster without a scaffold.

In some embodiments, transdifferentiated non-pancreatic beta insulin producing cells cultured as a 3D culture with a scaffold comprise increased insulin secretion compared to transdifferentiated non-pancreatic beta insulin producing cells cultured as a monolayer cell culture. In some embodiments, transdifferentiated non-pancreatic beta insulin producing cells cultured as a 3D culture with a scaffold comprise increased insulin secretion compared to transdifferentiated non-pancreatic beta insulin producing cells cultured as a 3D cell cluster without a scaffold. In some embodiments, transdifferentiated non-pancreatic beta insulin producing cells cultured as a 3D culture with a scaffold comprise increased insulin secretion compared to non-transdifferentiated non-pancreatic beta cells.

In some embodiments, transdifferentiated non-pancreatic beta insulin producing cells cultured as a 3D culture with a scaffold comprise increased C-peptide secretion compared to transdifferentiated non-pancreatic beta insulin producing cells cultured as a monolayer cell culture. In some embodiments, transdifferentiated non-pancreatic beta insulin producing cells cultured as a 3D culture with a scaffold comprise increased C-peptide secretion compared to transdifferentiated non-pancreatic beta insulin producing cells cultured as a 3D cell cluster without a scaffold. In some embodiments, transdifferentiated non-pancreatic beta insulin producing cells cultured as a 3D culture with a scaffold comprise increased C-peptide secretion compared to non-transdifferentiated non-pancreatic beta cells.

In some embodiments, transdifferentiated non-pancreatic beta insulin producing cells cultured as a 3D cluster with a scaffold comprise increased insulin content compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased insulin content compared to transdifferentiated non-pancreatic beta insulin producing cells cultured as a 3D cell cluster without a scaffold. In some embodiments, transdifferentiated non-pancreatic beta insulin producing cells cultured as a 3D cluster with a scaffold comprise increased insulin content compared to non-transdifferentiated non-pancreatic beta cells.

In some embodiments, transdifferentiated non-pancreatic beta insulin producing cells cultured as a 3D cluster with a scaffold comprise increased expression of GCG compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of GCG compared to transdifferentiated non-pancreatic beta insulin producing cells cultured as a 3D cell cluster without a scaffold. In some embodiments, transdifferentiated non-pancreatic beta insulin producing cells cultured as a 3D cluster with a scaffold comprise increased expression of GCG compared to non-transdifferentiated non-pancreatic beta cells.

In some embodiments, said increased expression of GCG is less than 10%. In some embodiments, said increased expression of GCG is between about 10% to 100%. In some embodiments, said increased expression of GCG is between about 200% to 300%. In some embodiments, said increased expression of GCG is between about 300% to 400%. In some embodiments, said increased expression of GCG is between about 400% to 500%. In some embodiments, said increased expression of GCG is between about 500% to 600%. In some embodiments, said increased expression of GCG is between about 600% to 700%. In some embodiments, said increased expression of GCG is between about 700% to 800%. In some embodiments, said increased expression of GCG is between about 800% to 900%. In some embodiments, said increased expression of GCG is between about 900% to 1000%. In some embodiments, said increased expression of GCG is between above 1000%.

In some embodiments, transdifferentiated non-pancreatic beta insulin producing cells cultured as a 3D cluster with a scaffold comprise increased expression of NKX6.1 compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of NKX6.1 compared to transdifferentiated non-pancreatic beta insulin producing cells cultured as a 3D cell cluster without a scaffold. In some embodiments, transdifferentiated non-pancreatic beta insulin producing cells cultured as a 3D cluster with a scaffold comprise increased expression of NKX6.1 compared to non-transdifferentiated non-pancreatic beta cells.

In some embodiments, said increased expression of NKX6.1 is less than 2-fold. In some embodiments, said increased expression of NKX6.1 is between about 2-fold to 5-fold. In some embodiments, said increased expression of NKX6.1 is between about 5-fold to 10-fold. In some embodiments, said increased expression of NKX6.1 is between about 10-fold to 20-fold. In some embodiments, said increased expression of NKX6.1 is between about 20-fold to 30-fold. In some embodiments, said increased expression of NKX6.1 is between about 30-fold to 40-fold. In some embodiments, said increased expression of NKX6.1 is between about 40-fold to 50-fold. In some embodiments, said increased expression of NKX6.1 is between about 50-fold to 60-fold. In some embodiments, said increased expression of NKX6.1 is between about 60-fold to 70-fold. In some embodiments, said increased expression of NKX6.1 is between about 70-fold to 80-fold. In some embodiments, said increased expression of NKX6.1 is between about 80-fold to 90-fold. In some embodiments, said increased expression of NKX6.1 is between about 90-fold to 100-fold. In some embodiments, said increased expression of NKX6.1 is above 100-fold.

In some embodiments, transdifferentiated non-pancreatic beta insulin producing cells cultured as a 3D cluster with a scaffold comprise increased expression of PAX6 compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of PAX6 compared to transdifferentiated non-pancreatic beta insulin producing cells cultured as a 3D cell cluster without a scaffold. In some embodiments, transdifferentiated non-pancreatic beta insulin producing cells cultured as a 3D cluster with a scaffold comprise increased expression of PAX6 compared to non-transdifferentiated non-pancreatic beta cells.

In some embodiments, said increased expression of PAX6 is less than 10%. In some embodiments, said increased expression of PAX6 is between about 10% to 100%. In some embodiments, said increased expression of PAX6 is between about 200% to 300%. In some embodiments, said increased expression of PAX6 is between about 300% to 400%. In some embodiments, said increased expression of PAX6 is between about 400% to 500%. In some embodiments, said increased expression of PAX6 is between about 500% to 600%. In some embodiments, said increased expression of PAX6 is between about 600% to 700%. In some embodiments, said increased expression of PAX6 is between about 700% to 800%. In some embodiments, said increased expression of PAX6 is between about 800% to 900%. In some embodiments, said increased expression of PAX6 is between about 900% to 1000%. In some embodiments, said increased expression of PAX6 is between above 1000%.

In some embodiments, the transdifferentiated non-pancreatic beta insulin producing cells cultured as a 3D cluster with a scaffold secrete at least 2 pm C-peptide/10⁶ cells/hour in response to high glucose concentrations. In some embodiments, the transdifferentiated non-pancreatic beta insulin producing cells cultured as a 3D cluster with a scaffold secrete at least 5 pm C-peptide/10⁶ cells/hour in response to high glucose concentrations. In some embodiments, the transdifferentiated non-pancreatic beta insulin producing cells cultured as a 3D cluster with a scaffold secrete at least 10 pm C-peptide/10⁶ cells/hour in response to high glucose concentrations. In some embodiments, the transdifferentiated non-pancreatic beta insulin producing cells cultured as a 3D cluster with a scaffold secrete at least 20 pm C-peptide/10⁶ cells/hour in response to high glucose concentrations. In some embodiments, the transdifferentiated non-pancreatic beta insulin producing cells cultured as a 3D cluster with a scaffold secrete at least 50 pm C-peptide/10⁶ cells/hour in response to high glucose concentrations. In some embodiments, the transdifferentiated non-pancreatic beta insulin producing cells cultured as a 3D cluster with a scaffold secrete at least 100 pm C-peptide/10⁶ cells/hour in response to high glucose concentrations. In some embodiments, the transdifferentiated non-pancreatic beta insulin producing cells cultured as a 3D cluster with a scaffold secrete at least 200 pm C-peptide/10⁶ cells/hour in response to high glucose concentrations. In some embodiments, the transdifferentiated non-pancreatic beta insulin producing cells cultured as a 3D cluster with a scaffold secrete at least 500 pm C-peptide/10⁶ cells/hour in response to high glucose concentrations. In some embodiments, the transdifferentiated non-pancreatic beta insulin producing cells cultured as a 3D cluster with a scaffold secrete at least 1000 pm C-peptide/10⁶ cells/hour in response to high glucose concentrations.

In some embodiments, glucose regulated insulin secretion comprises at least 0.001 pg insulin/10⁶ cells/hour in response to high glucose concentrations. In another embodiment, glucose regulated insulin secretion comprises at least 0.002 pg insulin/10⁶ cells/hour in response to high glucose concentrations. In another embodiment, glucose regulated insulin secretion comprises at least 0.003 pg insulin/10⁶ cells/hour in response to high glucose concentrations. In another embodiment, glucose regulated insulin secretion comprises at least 0.005 pg insulin/10⁶ cells/hour in response to high glucose concentrations. In another embodiment, glucose regulated insulin secretion comprises at least 0.007 pg insulin/10⁶ cells/hour in response to high glucose concentrations. In another embodiment, glucose regulated insulin secretion comprises at least 0.01 pg insulin/10⁶ cells/hour in response to high glucose concentrations. In another embodiment, glucose regulated insulin secretion comprises at least 0.1 pg insulin/10⁶ cells/hour in response to high glucose concentrations. In another embodiment, glucose regulated insulin secretion comprises at least 0.5 pg insulin/10⁶ cells/hour in response to high glucose concentrations. In another embodiment, glucose regulated insulin secretion comprises at least 1 pg insulin/10⁶ cells/hour in response to high glucose concentrations. In another embodiment, glucose regulated insulin secretion comprises at least 5 pg insulin/10⁶ cells/hour in response to high glucose concentrations. In another embodiment, glucose regulated insulin secretion comprises at least 10 pg insulin/10⁶ cells/hour in response to high glucose concentrations. In another embodiment, glucose regulated insulin secretion comprises at least 50 pg insulin/10⁶ cells/hour in response to high glucose concentrations. In another embodiment, glucose regulated insulin secretion comprises at least 100 pg insulin/10⁶ cells/hour in response to high glucose concentrations. In another embodiment, glucose regulated insulin secretion comprises at least 500 pg insulin/10⁶ cells/hour in response to high glucose concentrations. In another embodiment, glucose regulated insulin secretion comprises at least 1 ng insulin/10⁶ cells/hour in response to high glucose concentrations. In another embodiment, glucose regulated insulin secretion comprises at least 5 ng insulin/10⁶ cells/hour in response to high glucose concentrations. In another embodiment, glucose regulated insulin secretion comprises at least 10 ng insulin/10⁶ cells/hour in response to high glucose concentrations. In another embodiment, glucose regulated insulin secretion comprises at least 50 ng insulin/10⁶ cells/hour in response to high glucose concentrations. In another embodiment, glucose regulated insulin secretion comprises at least 100 ng insulin/10⁶ cells/hour in response to high glucose concentrations. In another embodiment, glucose regulated insulin secretion comprises at least 500 ng insulin/10⁶ cells/hour in response to high glucose concentrations. In another embodiment, glucose regulated insulin secretion comprises at least 1 μg insulin/10⁶ cells/hour in response to high glucose concentrations. In another embodiment, glucose regulated insulin secretion comprises at least 5 μg insulin/10⁶ cells/hour in response to high glucose concentrations. In another embodiment, glucose regulated insulin secretion comprises at least 10 μg insulin/10⁶ cells/hour in response to high glucose concentrations. In another embodiment, glucose regulated insulin secretion comprises at least 50 μg insulin/10⁶ cells/hour in response to high glucose concentrations. In another embodiment, glucose regulated insulin secretion comprises at least 100 μg insulin/10⁶ cells/hour in response to high glucose concentrations.

In some embodiments, a high glucose concentration comprises a concentration above 2 mM. In some embodiments, a high glucose concentration comprises a concentration above 5 mM. In some embodiments, a high glucose concentration comprises a concentration above 10 mM. In some embodiments, a high glucose concentration comprises a concentration above 15 mM. In some embodiments, a high glucose concentration comprises a concentration above 17.5 mM. In some embodiments, a high glucose concentration comprises a concentration above 20 mM.

In some embodiments, the transdifferentiated non-pancreatic beta insulin producing cells cultured as a 3D cluster with a scaffold comprise increased expression of the ectopic pancreatic transcription factors used for transdifferentiation compared to transdifferentiated non-pancreatic beta insulin producing cells transdifferentiated with similar ectopic pancreatic transcription factors and cultured as a monolayer cell culture. In some embodiments, the transdifferentiated non-pancreatic beta insulin producing cells cultured as a 3D cluster with a scaffold comprise increased expression of the ectopic pancreatic transcription factors used for transdifferentiation compared to transdifferentiated non-pancreatic beta insulin producing cells transdifferentiated with similar ectopic pancreatic transcription factors and cultured as a 3D cell cluster without a scaffold. In some embodiments, the ectopic pancreatic transcription factors are selected from PDX1, NeuroD1, Pax4 and/or MafA or any combination thereof.

In some embodiments, the expression of ectopic PDX1 is increased by at least 25% compared to the cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic PDX1 is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

In some embodiments, the expression of ectopic NeuroD1 is increased by at least 25% in transdifferentiated non-pancreatic beta insulin producing cells cultured as a 3D cluster with a scaffold compared to transdifferentiated cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic NeuroD1 is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

In some embodiments, the expression of ectopic MafA is increased by at least 25% in transdifferentiated non-pancreatic beta insulin producing cells cultured as a 3D cluster with a scaffold compared to transdifferentiated cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic MafA is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

A skilled artisan would appreciate that the term “monolayer cell culture” encompasses a type of culture in which no cell is growing on top of another, but all are growing side by side and often touching each other on the same growth surface. The term “monolayer cell culture” may be used interchangeably with “2D cell culture” having all the same qualities and meanings.

In some embodiments, the transdifferentiated non-pancreatic beta insulin producing cells cultured as a 3D cluster with a scaffold have increased viability compared to transdifferentiated non-pancreatic beta insulin producing cells cultured as a monolayer cell culture. In some embodiments, the transdifferentiated cells have similar viability than transdifferentiated non-pancreatic beta insulin producing cells cultured as a monolayer cell culture. In some embodiments, the transdifferentiated non-pancreatic beta insulin producing cells cultured as a 3D cluster with a scaffold have increased viability compared to transdifferentiated non-pancreatic beta insulin producing cells cultured as a 3D cell cluster without a scaffold. In some embodiments, the transdifferentiated non-pancreatic beta insulin producing cells cultured as a 3D cluster with a scaffold have similar viability than transdifferentiated non-pancreatic beta insulin producing cells cultured as a 3D cell cluster without a scaffold.

In some embodiments, the adult mammalian non-pancreatic beta cells are epithelial cells, endothelial cells, keratinocytes, fibroblasts, muscle cells, hepatocytes, liver cells, blood cells, stem or progenitor cells, liver stem cells, neural stem cells, mesenchymal stem cells, hematopoietic stem or progenitor cells, or any combination thereof. On one embodiment, the cell is totipotent or pluripotent. In some embodiments, the cell is an induced pluripotent stem cells. In some embodiments, stem or progenitor cells are obtained from bone marrow, umbilical cord blood, peripheral blood, fetal liver, adipose tissue, or any combination thereof. In some embodiments, the mammalian non-pancreatic beta cells are a combination of different cell types.

In some embodiments, the source of a cell population disclosed here is a human source. In another embodiment, the source of a cell population disclosed here in is an autologous human source relative to a subject in need of insulin therapy. In another embodiment, the source of a cell population disclosed here in is an allogeneic human source relative to a subject in need of insulin therapy.

In certain embodiments, the cell is a mesenchymal stem cell, also known as a mesenchymal stromal cell, derived from, liver tissue, adipose tissue, bone marrow, skin, placenta, umbilical cord, Wharton's jelly or cord blood. By “umbilical cord blood” or “cord blood” is meant to refer to blood obtained from a neonate or fetus, most preferably a neonate and preferably refers to blood which is obtained from the umbilical cord or the placenta of newborns. These cells can be obtained according to any conventional method known in the art. MSC are defined by expression of certain cell surface markers including, but not limited to, CD105, CD73 and CD90 and ability to differentiate into multiple lineages including osteoblasts, adipocytes and chondroblasts. MSC can be obtained from tissues by conventional isolation techniques such as plastic adherence, separation using monoclonal antibodies such as STRO-1 or through epithelial cells undergoing an epithelial-mesenchymal transition (EMT).

A skilled artisan would appreciate that the term “adipose tissue-derived mesenchymal stem cells” may encompass undifferentiated adult stem cells isolated from adipose tissue and may also be term “adipose stem cells”, having all the same qualities and meanings. These cells can be obtained according to any conventional method known in the art.

A skilled artisan would appreciate that the term, “placental-derived mesenchymal stem cells” may encompass undifferentiated adult stem cells isolated from placenta and may be referred to herein as “placental stem cells”, having all the same meanings and qualities.

In some embodiments, cell population that is exposed to, i.e., contacted with, the compounds (i.e. PDX-1, Pax-4, MafA, NeuroD1 and/or Sox-9 polypeptides or nucleic acid encoding PDX-1, Pax-4, MafA, NeuroD1 and/or Sox-9 polypeptides) can be any number of cells, i.e., one or more cells, and can be provided in vitro, in vivo, or ex vivo. The cell population that is contacted with the transcription factors can be expanded in vitro prior to being contacted with the transcription factors. The cell population produced exhibits a mature pancreatic beta cell phenotype. These cells can be expanded in vitro by methods known in the art prior to transdifferentiation and maturation along the β-cell lineage, and prior to administration or delivery to a patient or subject in need thereof.

Therapeutics Compositions

The herein-described tridimensional (3D) clusters of transdifferentiated cells wherein at least a subset of said cells are attached to a scaffold, when used therapeutically, are referred to herein as “therapeutics”. Methods of administration of therapeutics include, but are not limited to, intradermal, intraperitoneal, intravenous, surgical as an implant, and oral routes. The therapeutics of the disclosure presented herein may be administered by any convenient route, for example by infusion, by bolus injection, by surgical implantation and may be administered together with other biologically-active agents. Administration can be systemic or local, e.g. through portal vein delivery to the liver, or to the pancreas It may also be desirable to administer the therapeutic locally to the area in need of treatment; this may be achieved by, for example, and not by way of limitation, local infusion during surgery, by injection, by means of a catheter, or by means of an implant. In some embodiments, the therapeutic is administered intravenously. Specifically, the therapeutic can be delivered via a portal vein infusion.

A skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

Suitable dosage ranges for intravenous administration of the therapeutics of the disclosure presented herein are generally at least 1 million transdifferentiated cells, at least 2 million transdifferentiated cells, at least 5 million transdifferentiated cells, at least 10 million transdifferentiated cells, at least 25 million transdifferentiated cells, at least 50 million transdifferentiated cells, at least 100 million transdifferentiated cells, at least 200 million transdifferentiated cells, at least 300 million transdifferentiated cells, at least 400 million transdifferentiated cells, at least 500 million transdifferentiated cells, at least 600 million transdifferentiated cells, at least 700 million transdifferentiated cells, at least 800 million transdifferentiated cells, at least 900 million transdifferentiated cells, at least 1 billion transdifferentiated cells, at least 2 billion transdifferentiated cells, at least 3 billion transdifferentiated cells, at least 4 billion transdifferentiated cells, or at least 5 billion transdifferentiated cells. In some embodiments, the dose is 1-2 billion transdifferentiated cells into a 60-75 kg subject. One skilled in the art would appreciate that effective doses may be extrapolated from dose-response curves derived from in vitro or animal model test systems. In another embodiment, the effective dose may be administered intravenously into the liver portal vein.

Cells may also be cultured ex vivo in the presence of therapeutics of the disclosure presented herein in order to proliferate or to produce a desired effect on or activity in such cells. Treated cells can then be introduced in vivo via the administration routes described herein for therapeutic purposes.

Pharmaceutical Compositions

The herein-described tridimensional (3D) clusters of transdifferentiated cells, wherein at least a subset of said cells are attached to a scaffold, can be incorporated into pharmaceutical compositions suitable for administration. Such compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition disclosed here is formulated to be compatible with its intended route of administration. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

Methods of Generating Three-Dimensional (3D) Cell Clusters

Disclosed herein are methods of generating a 3D cell cluster of transdifferentiated mammalian non-pancreatic beta insulin producing cells, wherein at least a subset of said transdifferentiated cells are attached to a scaffold. In some embodiments, the methods comprise propagating, expanding, transdifferentiating and attaching the cells to a scaffold. In some embodiments, a pancreatic beta cell phenotype comprises a mature pancreatic beta cell phenotype.

In some embodiments, the cells are obtained from a human tissue. In some embodiments, the human tissue is processed to recover primary human non-pancreatic cells. In some embodiments, cells are seeded on a scaffold and propagated and/or expanded on it. In some embodiments, cells are transdifferentiated while being attached to a scaffold. In some embodiments, cells are attached to a scaffold following transdifferentiation. In some embodiments, cells are propagated and/or expanded under non-adherent cell culture conditions. In some embodiments, cells are transdifferentiated under non-adherent conditions.

A skilled artisan would appreciate that the term “non-adherent cell culture conditions” encompasses a type of culture in which single cells or small aggregates of cells are grown while suspended in a liquid medium, and that the term may be used interchangeably with “cell suspension culture” having the same qualities and meanings.

In some embodiments, cells can be grown under non-adherent conditions as a batch culture, i.e., growing in a closed system having a specific volume of agitated medium, with no additions of nutrients or removal of waste products. Batch cultures can be maintained in a recipient such as flasks, conical flasks, or well plates mounted on orbital platform shakers. Alternatively, batch cultures can be maintained in nipple flasks, that alternative expose the cells to the medium and to air. Alternatively, batch cultures can be maintained in spinning cultures, consisting of large bottles containing volumes of medium of about 10 liters that spin around their axis at a predetermined speed and are usually tilted in a predetermined angle. Alternatively, batch cultures can be maintained in stirred cultures, consisting of large culture vessels containing medium into which sterile air is bubbled and/or is agitated by stirrers.

In some embodiments, cells can be grown under non-adherent conditions in continuous culture, i.e., a system in which medium is replaced as to provide cells with nutrients and remove waste. Continuous culture can be closed type, i.e, a system in which the cells are retrieved and added back to the culture. Continuous culture can be open type, i.e., both cells and medium are replaced with fresh medium. Open continuous culture can be carried in a chemostat bioreactor, i.e., a bioreactor to which fresh medium is continuously added, while the present medium is continuously removed at the same rate. Open continuous culture can be carried in a turbidostat, which dynamically adjusts the medium flow rate according to the cell concentration in the medium as determined by medium turbidity. Open continuous culture can be carried in an auxostat, which dynamically adjusts the medium flow rate according to a measurement taken, such as pH, oxygen, ethanol concentrations, sugar concentrations, etc.

In some embodiments, 3D clusters attached to a scaffold can be grown in a bioreactor. A skilled artisan would appreciate that a bioreactor can simulate IPC physiological environment in order to promote cell survival, proliferation, or a pancreatic β cell like phenotype. The physiological environment can comprise parameters as temperature, oxygen concentration, carbon dioxide concentration, or any other relevant biological, chemical or mechanical stimuli. In some instances, the bioreactor comprises one or more small plastic cylindrical chambers with monitored temperature and humidity conditions suitable for growing 3D clusters. The bioreactor can also use bioactive synthetic materials such as polyethylene terephthalate membranes to surround the 3D clusters in a closed environment into which any soluble factors of interest can be provided. The chambers of the bioreactor can rotate as to ensure equal cell growth in all directions.

In some embodiments, at least a subset of the primary cells is attached to a scaffold. In some embodiments, at least a subset of the propagated and expanded cells is attached to a scaffold. In some embodiments, at least a subset of the transdifferentiated cells is attached to a scaffold.

Methods for transdifferentiating cells are described in U.S. Pat. No. 6,774,120, U.S. Publication No. 2005/0090465, U.S. Publication No. 2016/0220616, all the contents of which are incorporated by reference in their entireties. In some embodiments, the methods comprise contacting mammalian non-pancreatic cells with pancreatic transcription factors, such as PDX-1, Pax-4, NeuroD1, and MafA, at specific time points. In some embodiments, the methods comprise contacting a mammalian non-pancreatic cell with PDX-1 at a first timepoint; contacting the cells from the first step with Pax-4 at a second timepoint; and contacting the cells from the second step with MafA at a third timepoint. In some embodiments, the methods comprise contacting a mammalian non-pancreatic cell with PDX-1 at a first timepoint; contacting the cells from the first step with NeuroD1 at a second timepoint; and contacting the cells from the second step with MafA at a third timepoint. In another embodiment, the methods comprise contacting a mammalian non-pancreatic cell with PDX-1 and a second transcription factor at a first timepoint and contacting the cells from the first step with MafA at a second timepoint. In yet a further embodiment, a second transcription factor is selected from NeuroD1 and Pax4. In another embodiment, the transcription factors provided together with PDX-1 comprise Pax-4, NeuroD1, Ngn3, or Sox-9. In another embodiment, the transcription factors provided together with PDX-1 comprises Pax-4. In another embodiment, the transcription factors provided together with PDX-1 comprises NeuroD1. In another embodiment, the transcription factors provided together with PDX-1 comprises Ngn3. In another embodiment, the transcription factors provided together with PDX-1 comprises Sox-9.

In other embodiments, the methods comprise contacting a mammalian non-pancreatic cell with PDX-1 at a first timepoint; contacting the cells from the first step with Ngn3 at a second timepoint; and contacting the cells from the second step with MafA at a third timepoint. In other embodiments, the methods comprise contacting a mammalian non-pancreatic cell with PDX-1 at a first timepoint; contacting the cells from the first step with Sox9 at a second timepoint; and contacting the cells from the second step with MafA at a third timepoint. In another embodiment, the methods comprise contacting a mammalian non-pancreatic cell with PDX-1 and a second transcription factor at a first timepoint and contacting the cells from the first step with MafA at a second timepoint, wherein a second transcription factor is selected from NeuroD1, Ngn3, Sox9, and Pax4.

In another embodiment, the methods comprise contacting a mammalian non-pancreatic cell with PDX-1 and NeuroD1 at a first timepoint, and contacting the cells from the first step with MafA at a second timepoint. In another embodiment, the methods comprise contacting a mammalian non-pancreatic cell with PDX-1 and Pax4 at a first timepoint, and contacting the cells from the first step with MafA at a second timepoint. In another embodiment, the methods comprise contacting a mammalian non-pancreatic cell with PDX-1 and Ngn3 at a first timepoint, and contacting the cells from the first step with MafA at a second timepoint. In another embodiment, the methods comprise contacting a mammalian non-pancreatic cell with PDX-1 and Sox9 at a first timepoint, and contacting the cells from the first step with MafA at a second timepoint.

In another embodiment, the cells are contacted with all three factors (PDX-1; NeuroD1 or Pax4 or Ngn3; and MafA) at the same time but their expression levels are controlled in such a way as to have them expressed within the cell at a first timepoint for PDX-1, a second timepoint for NeuroD1 or Pax4 or Ngn3; and a third timepoint for MafA. The control of expression can be achieved by using appropriate promoters on each gene such that the genes are expressed sequentially, by modifying levels of mRNA, or by other means known in the art.

In some embodiments, the methods described herein further comprise contacting the cells at, before, or after any of the above steps with the transcription factor Sox-9.

In some embodiments, the first and second timepoints are identical resulting in contacting a cell population with two pTFs at a first timepoint, wherein at least one pTF comprises PDX-1, followed by contacting the resultant cell population with a third pTF at a second timepoint, wherein said third pTF is MafA.

The cell population that is exposed to, i.e., contacted with, the compounds (i.e. PDX-1, Pax-4, MafA, NeuroD1 and/or Sox-9 polypeptides or nucleic acid encoding PDX-1, Pax-4, MafA, NeuroD1 and/or Sox-9 polypeptides) can be any number of cells, i.e., one or more cells, and can be provided in vitro, in vivo, or ex vivo. The cell population that is contacted with the transcription factors can be expanded in vitro prior to being contacted with the transcription factors. The cell population produced exhibits a mature pancreatic beta cell phenotype. These cells can be expanded in vitro by methods known in the art prior to transdifferentiation and maturation along the □-cell lineage, and prior to administration or delivery to a patient or subject in need thereof.

In some embodiments, the second timepoint is at least 24 hours after the first timepoint. In an alternative embodiment, the second timepoint is less than 24 hours after the first timepoint. In another embodiment, the second timepoint is about 1 hour after the first timepoint, about 2 hours, about 3 hours, about 4 hours, about 5 hours, about 6 hours, about 7 hours, about 8 hours, about 9 hours, about 10 hours, about 11 hours, or about 12 hours after the first timepoint. In some embodiments, the second timepoint can be at least 24 hours, at least 48 hours, at least 72 hours, and at least 1 week or more after the first timepoint.

In another embodiment, the third timepoint is at least 24 hours after the second timepoint. In an alternative embodiment, the third timepoint is less than 24 hours after the second timepoint. In another embodiment, the third timepoint is at the same time as the second timepoint. In another embodiment, the third timepoint is about 1 hour after the second timepoint, about 2 hours, about 3 hours, about 4 hours, about 5 hours, about 6 hours, about 7 hours, about 8 hours, about 9 hours, about 10 hours, about 11 hours, or about 12 hours after the second timepoint. In other embodiments, the third timepoint can be at least 24 hours, at least 48 hours, at least 72 hours, and at least 1 week or more after the second timepoint.

In some embodiments, the first, second, and third timepoints are concurrent resulting in contacting a cell population with three pTFs at a single timepoint, wherein at least one pTF comprises PDX-1, at least one pTF comprises NeuroD1 or Pax4, and at least one pTF comprises MafA. In another embodiment, the first, second, and third timepoints are concurrent resulting in contacting a cell population with three pTFs at a single timepoint, wherein one pTF consists of PDX-1, one pTF consists of NeuroD1 or Pax4, and one pTF consists of MafA. A skilled artisan would appreciate that the term “timepoint” comprises a point in time, or a specific instant. In some embodiments, a timepoint comprises a short lapse of time. In some embodiments, a timepoint comprises less than 24 hours. In some embodiments, a timepoint comprises less than 12 hours. In some embodiments, a timepoint comprises less than 6 hours. In some embodiments, a timepoint comprises less than 3 hours. In some embodiments, a timepoint comprises less than 1 hour. In some embodiments, a timepoint comprises less than 30 minutes. In some embodiments, a timepoint comprises less than 10 minutes. In some embodiments, a timepoint comprises less than 5 minutes. In some embodiments, a timepoint comprises less than 1 minute. In some embodiments, a timepoint comprises less than 10 seconds.

In some embodiments, transcription factors comprise polypeptides, or ribonucleic acids or nucleic acids encoding the transcription factor polypeptides. In another embodiment, the transcription factor comprises a polypeptide. In another embodiment, the transcription factor comprises a nucleic acid sequence encoding the transcription factor. In another embodiment, the transcription factor comprises a Deoxyribonucleic acid sequence (DNA) encoding the transcription factor. In still another embodiment, the DNA comprises a cDNA. In another embodiment, the transcription factor comprises a ribonucleic acid sequence (RNA) encoding the transcription factor. In yet another embodiment, the RNA comprises an mRNA.

Transcription factors for use in the disclosure presented herein can be a polypeptide, ribonucleic acid or a nucleic acid. A skilled artisan would appreciate that the term “nucleic acid” may encompass DNA molecules (e.g., cDNA or genomic DNA), RNA molecules (e.g., mRNA, microRNA or other RNA derivatives), analogs of the DNA or RNA generated using nucleotide analogs, and derivatives, fragments and homologs thereof. The nucleic acid molecule can be single-stranded or double-stranded. In some embodiments, the nucleic acid is a DNA. In other embodiments, the nucleic acid is mRNA. mRNA is particularly advantageous in the methods disclosed herein, as transient expression of PDX-1 is sufficient to produce pancreatic beta cells. The polypeptide, ribonucleic acid or nucleic acid maybe delivered to the cell by means known in the art including, but not limited to, infection with viral vectors, electroporation and lipofection.

In some embodiments, the polypeptide, ribonucleic acid or nucleic acid is delivered to the cell by a viral vector. In some embodiments, the ribonucleic acid or nucleic acid is incorporated in an expression vector or a viral vector. In some embodiments, the viral vector is an adenovirus vector. In another embodiment, an adenoviral vector is a first generation adenoviral (FGAD) vector. In another embodiment, an FGAD is unable in integrate into the genome of a cell. In another embodiment, a FGAD comprises an E1-deleted recombinant adenoviral vector. In another embodiment, a FGAD provide transient expression of encoded polypeptides.

The expression or viral vector can be introduced to the cell by any of the following: transfection, electroporation, infection, or transduction. In other embodiments, the nucleic acid is mRNA and it is delivered for example by electroporation. In some embodiments, methods of electroporation comprise flow electroporation technology. For example, in another embodiment, methods of electroporation comprise use of a MaxCyte electroporation system (MaxCyte Inc. Georgia USA).

In certain embodiments, transcription factors for use in the methods described herein are selected from the group consisting of PDX-1, Pax-4, NeuroD1, and MafA. In other embodiments, transcription factors for use in the methods described herein are selected from the group consisting of PDX-1, Pax-4, NeuroD1, MafA, Ngn3, and Sox9.

The homeodomain protein PDX-1 (pancreatic and duodenal homeobox gene-1), also known as IDX-1, IPF-1, STF-1, or IUF-1, plays a central role in regulating pancreatic islet development and function. PDX-1 is either directly or indirectly involved in islet-cell-specific expression of various genes such as, for example insulin, glucagon, somatostatin, proinsulin convertase 1/3 (PC1/3), GLUT-2 and glucokinase. Additionally, PDX-1 mediates insulin gene transcription in response to glucose. Suitable sources of nucleic acids encoding PDX-1 include for example the human PDX-1 nucleic acid (and the encoded protein sequences) available as GenBank Accession Nos. U35632 and AAA88820, respectively. In some embodiments, the amino acid sequence of a PDX-1 polypeptide is set forth in SEQ ID NO: 1:

(SEQ ID NO: 1)
MNGEEQYYAATQLYKDPCAFQRGPAPEFSASPPACLYMGRQPP
PPPPHPFPGALGALEQGSPPDISPYEVPPLADDPAVAHLHHHL
PAQLALPHPPAGPFPEGAEPGVLEEPNRVQLPFPWMKSTKAHA
WKGQWAGGAYAAEPEENKRTRTAYTRAQLLELEKEFLFNKYIS
RPRRVELAVMLNLTERHIKIWFQNRRMKWKKEEDKKRGGGTAV
GGGGVAEPEQDCAVTSGEELLALPPPPPPGGAVPPAAPVAARE
GRLPPGLSASPQPSSVAPRRPQEPR.

In some embodiments, the nucleic acid sequence of a PDX-1 polynucleotide is set forth in SEQ ID NO: 2:

(SEQ ID NO: 2)
ATGAACGGCGAGGAGCAGTACTACGCGGCCACGCAGCTTTACA
AGGACCCATGCGCGTTCCAGCGAGGCCCGGCGCCGGAGTTCAG
CGCCAGCCCCCCTGCGTGCCTGTACATGGGCCGCCAGCCCCCG
CCGCCGCCGCCGCACCCGTTCCCTGGCGCCCTGGGCGCGCTGG
AGCAGGGCAGCCCCCCGGACATCTCCCCGTACGAGGTGCCCCC
CCTCGCCGACGACCCCGCGGTGGCGCACCTTCACCACCACCTC
CCGGCTCAGCTCGCGCTCCCCCACCCGCCCGCCGGGCCCTTCC
CGGAGGGAGCCGAGCCGGGCGTCCTGGAGGAGCCCAACCGCGT
CCAGCTGCCTTTCCCATGGATGAAGTCTACCAAAGCTCACGCG
TGGAAAGGCCAGTGGGCAGGCGGCGCCTACGCTGCGGAGCCGG
AGGAGAACAAGCGGACGCGCACGGCCTACACGCGCGCACAGCT
GCTAGAGCTGGAGAAGGAGTTCCTATTCAACAAGTACATCTCA
CGGCCGCGCCGGGTGGAGCTGGCTGTCATGTTGAACTTGACCG
AGAGACACATCAAGATCTGGTTCCAAAACCGCCGCATGAAGTG
GAAAAAGGAGGAGGACAAGAAGCGCGGCGGCGGGACAGCTGTC
GGGGGTGGCGGGGTCGCGGAGCCTGAGCAGGACTGCGCCGTGA
CCTCCGGCGAGGAGCTTCTGGCGCTGCCGCCGCCGCCGCCCCC
CGGAGGTGCTGTGCCGCCCGCTGCCCCCGTTGCCGCCCGAGAG
GGCCGCCTGCCGCCTGGCCTTAGCGCGTCGCCACAGCCCTCCA
GCGTCGCGCCTCGGCGGCCGCAGGAACCACGATGA.

Other sources of sequences for PDX-1 include rat PDX nucleic acid and protein sequences as shown in GenBank Accession No. U35632 and AAA18355, respectively, and are incorporated herein by reference in their entirety. An additional source includes zebrafish PDX-1 nucleic acid and protein sequences are shown in GenBank Accession No. AF036325 and AAC41260, respectively, and are incorporated herein by reference in their entirety.

Pax-4, also known as paired box 4, paired box protein 4, paired box gene 4, MODY9 and KPD, is a pancreatic-specific transcription factor that binds to elements within the glucagon, insulin and somatostatin promoters, and is thought to play an important role in the differentiation and development of pancreatic islet beta cells. In some cellular contexts, Pax-4 exhibits repressor activity. Suitable sources of nucleic acids encoding Pax-4 include for example the human Pax-4 nucleic acid (and the encoded protein sequences) available as GenBank Accession Nos. NM_006193.2 and AAD02289.1, respectively.

MafA, also known as V-maf musculoaponeurotic fibrosarcoma oncogene homolog A or RIPE3B1, is a beta-cell-specific and glucose-regulated transcriptional activator for insulin gene expression. MafA may be involved in the function and development of β cells as well as in the pathogenesis of diabetes. Suitable sources of nucleic acids encoding MafA include for example the human MafA nucleic acid (and the encoded protein sequences) available as GenBank Accession Nos. NM_201589.3 and NP_963883.2, respectively. In some embodiments, the amino acid sequence of a MafA polypeptide is set forth in SEQ ID NO: 3:

(SEQ ID NO: 3)
MAAELAMGAELPSSPLAIEYVNDFDLMKFEVKKEPPEAERFCH
RLPPGSLSSTPLSTPCSSVPSSPSFCAPSPGTGGGGGAGGGGG
SSQAGGAPGPPSGGPGAVGGTSGKPALEDLWMSGYQHHLNPEA
LNLTPEDAVEALIGSGHHGAHHGAHHPAAAAAYEAFRGPGFAG
GGGADDMGAGHHHGAHHAAHHHHAAHHHHHHHHHHGGAGHGGG
AGHHVRLEERFSDDQLVSMSVRELNRQLRGFSKEEVIRLKQKR
RTLKNRGYAQSCRFKRVQQRHILESEKCQLQSQVEQLKLEVGR
LAKERDLYKEKYEKLAGRGGPGSAGGAGFPREPSPPQAGPGGA
KGTADFFL.

In another embodiment, the nucleic acid sequence of a MafA polynucleotide is set forth in SEQ ID NO: 4:

(SEQ ID NO: 4)
ATGGCCGCGGAGCTGGCGATGGGCGCCGAGCTGCCCAGCAGCC
CGCTGGCCATCGAGTACGTCAACGACTTCGACCTGATGAAGTT
CGAGGTGAAGAAGGAGCCTCCCGAGGCCGAGCGCTTCTGCCAC
CGCCTGCCGCCAGGCTCGCTGTCCTCGACGCCGCTCAGCACGC
CCTGCTCCTCCGTGCCCTCCTCGCCCAGCTTCTGCGCGCCCAG
CCCGGGCACCGGCGGCGGCGGCGGCGCGGGGGGCGGCGGCGGC
TCGTCTCAGGCCGGGGGCGCCCCCGGGCCGCCGAGCGGGGGCC
CCGGCGCCGTCGGGGGCACCTCGGGGAAGCCGGCGCTGGAGGA
TCTGTACTGGATGAGCGGCTACCAGCATCACCTCAACCCCGAG
GCGCTCAACCTGACGCCCGAGGACGCGGTGGAGGCGCTCATCG
GCAGCGGCCACCACGGCGCGCACCACGGCGCGCACCACCCGGC
GGCCGCCGCAGCCTACGAGGCTTTCCGCGGCCCGGGCTTCGCG
GGCGGCGGCGGAGCGGACGACATGGGCGCCGGCCACCACCACG
GCGCGCACCACGCCGCCCACCACCACCACGCCGCCCACCACCA
CCACCACCACCACCACCATGGCGGCGCGGGACACGGCGGTGGC
GCGGGCCACCACGTGCGCCTGGAGGAGCGCTTCTCCGACGACC
AGCTGGTGTCCATGTCGGTGCGCGAGCTGAACCGGCAGCTCCG
CGGCTTCAGCAAGGAGGAGGTCATCCGGCTCAAGCAGAAGCGG
CGCACGCTCAAGAACCGCGGCTACGCGCAGTCCTGCCGCTTCA
AGCGGGTGCAGCAGCGGCACATTCTGGAGAGCGAGAAGTGCCA
ACTCCAGAGCCAGGTGGAGCAGCTGAAGCTGGAGGTGGGGCGC
CTGGCCAAAGAGCGGGACCTGTACAAGGAGAAATACGAGAAGC
TGGCGGGCCGGGGCGGCCCCGGGAGCGCGGGCGGGGCCGGTTT
CCCGCGGGAGCCTTCGCCGCCGCAGGCCGGTCCCGGCGGGGCC
AAGGGCACGGCCGACTTCTTCCTGTAG

Neurog3, also known as neurogenin 3 or Ngn3, is a basic helix-loop-helix (bHLH) transcription factor required for endocrine development in the pancreas and intestine. Suitable sources of nucleic acids encoding Neurog3 include for example the human Neurog3 nucleic acid (and the encoded protein sequences) available as GenBank Accession Nos. NM_020999.3 and NP_066279.2, respectively.

NeuroD1, also known as Neuro Differentiation 1 or NeuroD, and beta-2 (□ 2) is a Neuro D-type transcription factor. It is a basic helix-loop-helix transcription factor that forms heterodimers with other bHLH proteins and activates transcription of genes that contain a specific DNA sequence known as the E-box. It regulates expression of the insulin gene, and mutations in this gene result in type II diabetes mellitus. Suitable sources of nucleic acids encoding NeuroD1 include for example the human NeuroD1 nucleic acid (and the encoded protein sequences) available as GenBank Accession Nos. NM_002500.4 and NP_002491.2, respectively.

In some embodiments, the amino acid sequence of a NeuroD1 polypeptide is set forth in SEQ ID NO: 5:

(SEQ ID NO: 5)
MTKSYSESGLMGEPQPQGPPSWTDECLSSQDEEHEADKKEDDL
ETMNAEEDSLRNGGEEEDEDEDLEEEEEEEEEDDDQKPKRRGP
KKKKMTKARLERFKLRRMKANARERNRMHGLNAALDNLRKVVP
CYSKTQKLSKIETLRLAKNYIWALSEILRSGKSPDLVSFVQTL
CKGLSQPTTNLVAGCLQLNPRTFLPEQNQDMPPHLPTASASFP
VHPYSYQSPGLPSPPYGTMDSSHVFHVKPPPHAYSAALEPFFE
SPLTDCTSPSFDGPLSPPLSINGNFSFKHEPSAEFEKNYAFTM
HYPAATLAGAQSHGSIFSGTAAPRCEIPIDNIMSFDSHSHHER
VMSAQLNAIFHD.

In another embodiment, the nucleic acid sequence of a NeuroD1 polynucleotide is set forth in SEQ ID NO: 6:

(SEQ ID NO: 6)
ATGACCAAATCGTACAGCGAGAGTGGGCTGATGGGCGAGCCTC
AGCCCCAAGGTCCTCCAAGCTGGACAGACGAGTGTCTCAGTTC
TCAGGACGAGGAGCACGAGGCAGACAAGAAGGAGGACGACCTC
GAAGCCATGAACGCAGAGGAGGACTCACTGAGGAACGGGGGAG
AGGAGGAGGACGAAGATGAGGACCTGGAAGAGGAGGAAGAAGA
GGAAGAGGAGGATGACGATCAAAAGCCCAAGAGACGCGGCCCC
AAAAAGAAGAAGATGACTAAGGCTCGCCTGGAGCGTTTTAAAT
TGAGACGCATGAAGGCTAACGCCCGGGAGCGGAACCGCATGCA
CGGACTGAACGCGGCGCTAGACAACCTGCGCAAGGTGGTGCCT
TGCTATTCTAAGACGCAGAAGCTGTCCAAAATCGAGACTCTGC
GCTTGGCCAAGAACTACATCTGGGCTCTGTCGGAGATCTCGCG
CTCAGGCAAAAGCCCAGACCTGGTCTCCTTCGTTCAGACGCTT
TGCAAGGGCTTATCCCAACCCACCACCAACCTGGTTGCGGGCT
GCCTGCAACTCAATCCTCGGACTTTTCTGCCTGAGCAGAACCA
GGACATGCCCCCGCACCTGCCGACGGCCAGCGCTTCCTTCCCT
GTACACCCCTACTCCTACCAGTCGCCTGGGCTGCCCAGTCCGC
CTTACGGTACCATGGACAGCTCCCATGTCTTCCACGTTAAGCC
TCCGCCGCACGCCTACAGCGCAGCGCTGGAGCCCTTCTTTGAA
AGCCCTCTGACTGATTGCACCAGCCCTTCCTTTGATGGACCCC
TCAGCCCGCCGCTCAGCATCAATGGCAACTTCTCTTTCAAACA
CGAACCGTCCGCCGAGTTTGAGAAAAATTATGCCTTTACCATG
CACTATCCTGCAGCGACACTGGCAGGGGCCCAAAGCCACGGAT
CAATCTTCTCAGGCACCGCTGCCCCTCGCTGCGAGATCCCCAT
AGACAATATTATGTCCTTCGATAGCCATTCACATCATGAGCGA
GTCATGAGTGCCCAGCTCAATGCCATATTTCATGATTAG.

Sox9 is a transcription factor that is involved in embryonic development. Sox9 has been particularly investigated for its importance in bone and skeletal development. SOX-9 recognizes the sequence CCTTGAG along with other members of the HMG-box class DNA-binding proteins. In the context of the disclosure presented herein, the use of Sox9 may be involved in maintaining the pancreatic progenitor cell mass, either before or after induction of transdifferentiation. Suitable sources of nucleic acids encoding Sox9 include for example the human Sox9 nucleic acid (and the encoded protein sequences) available as GenBank Accession Nos. NM_000346.3 and NP_000337.1, respectively.

Homology is, in some embodiments, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-6 of greater than 60%. In another embodiment, “homology” refers to identity to a sequence selected from SEQ ID No: 1-6 of greater than 70%. In another embodiment, the identity is greater than 75%, greater than 78%, greater than 80%, greater than 82%, greater than 83%, greater than 85%, greater than 87%, greater than 88%, greater than 90%, greater than 92%, greater than 93%, greater than 95%, greater than 96%, greater than 97%, greater than 98%, or greater than 99%. In another embodiment, the identity is 100%. Each possibility represents a separate embodiment of the disclosure presented herein.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example, methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5×Denhardt's solution, 10% dextran sulfate, and 20 μg/ml denatured, sheared salmon sperm DNA.

Protein and/or peptide homology for any amino acid sequence listed herein is determined, in some embodiments, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the disclosure presented herein.

Another embodiment disclosed herein, pertains to vectors. In some embodiments, a vector used in the methods disclosed herein comprises an expression vector. In another embodiment, an expression vector comprises a nucleic acid encoding a PDX-1, Pax-4, NeuroD1 or MafA protein, or other pancreatic transcription factor, such as Ngn3, or derivatives, fragments, analogs, homologs or combinations thereof. In some embodiments, the expression vector comprises a single nucleic acid encoding any of the following transcription factors: PDX-1, Pax-4, NeuroD1, Ngn3, MafA, or Sox-9 or derivatives or fragments thereof. In some embodiments, the expression vector comprises two nucleic acids encoding any combination of the following transcription factors: PDX-1, Pax-4, NeuroD1, Ngn3, MafA, or Sox-9 or derivatives or fragments thereof. In a yet another embodiment, the expression vector comprises nucleic acids encoding PDX-1 and NeuroD1. In a still another embodiment, the expression vector comprises nucleic acids encoding PDX-1 and Pax4. In another embodiment, the expression vector comprises nucleic acids encoding MafA.

A skilled artisan would appreciate that the term “vector” encompasses a nucleic acid molecule capable of transporting another nucleic acid to which it has been linked. One type of vector is a “plasmid”, which encompasses a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. A skilled artisan would appreciate that the terms “plasmid” and “vector” may be used interchangeably having all the same qualities and meanings. In some embodiments, the term “plasmid” is the most commonly used form of vector. However, the disclosure presented herein is intended to include such other forms of expression vectors, such as viral vectors (e.g., replication defective retroviruses, lentivirus, adenoviruses and adeno-associated viruses), which serve equivalent functions. Additionally, some viral vectors are capable of targeting a particular cell type either specifically or non-specifically.

The recombinant expression vectors disclosed herein comprise a nucleic acid disclosed herein, in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, a skilled artisan would appreciate that the term “operably linked” may encompass nucleotide sequences of interest linked to the regulatory sequence(s) in a manner that allows for expression of the nucleotide sequence (e.g., in an in vitro transcription/translation system or in a host cell when the vector is introduced into the host cell). A skilled artisan would appreciate that term “regulatory sequence” may encompass promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors disclosed here may be introduced into host cells to thereby produce proteins or peptides, including fusion proteins or peptides, encoded by nucleic acids as described herein (e.g., PDX-1, Pax-4, MafA, NeuroD1 or Sox-9 proteins, or mutant forms or fusion proteins thereof, etc.).

For example, an expression vector comprises one nucleic acid encoding a transcription factor operably linked to a promoter. In expression vectors comprising two nucleic acids encoding transcription factors, each nucleic acid may be operably linked to a promoter. The promoter operably linked to each nucleic acid may be different or the same. Alternatively, the two nucleic acids may be operably linked to a single promoter. Promoters useful for the expression vectors disclosed here could be any promoter known in the art. The ordinarily skilled artisan could readily determine suitable promoters for the host cell in which the nucleic acid is to be expressed, the level of expression of protein desired, or the timing of expression, etc. The promoter may be a constitutive promoter, an inducible promoter, or a cell-type specific promoter.

The recombinant expression vectors disclosed here can be designed for expression of PDX-1 in prokaryotic or eukaryotic cells. For example, PDX-1, Pax-4, MafA, NeuroD1, and/or Sox-9 can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

In another embodiment, the PDX-1, Pax-4, MafA, NeuroD1, or Sox-9 expression vector is a yeast expression vector. Examples of vectors for expression in yeast S. cerevisiae include pYepSec1 (Baldari, et al., (1987) EMBO J 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, PDX-1, Pax-4, MafA, NeuroD1 or Sox-9 can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid disclosed here is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e g , milk whey promoter; U.S. Pat. No. 4,873,316 and European Application Publication No. 264,166). Developmentally regulated promoters are also encompassed, e.g., the murine hox promoters (Kessel and Gruss (1990) Science 249:374-379) and the alpha-fetoprotein promoter (Campes and Tilghman (1989) Genes Dev 3:537-546).

Another embodiment disclosed herein pertains to host cells into which a recombinant expression vector disclosed here has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Additionally, host cells could be modulated once expressing PDX-1, Pax-4, MafA, NeuroD1 or Sox-9 or a combination thereof, and may either maintain or loose original characteristics.

Vector DNA may be introduced into cells via conventional transformation, transduction, infection or transfection techniques. A skilled artisan would appreciate that the terms “transformation” “transduction”, “infection” and “transfection” may encompass a variety of art-recognized techniques for introducing foreign nucleic acid (e.g., DNA) into a host cell, including calcium phosphate or calcium chloride co-precipitation, DEAE-dextran-mediated transfection, lipofection, or electroporation. In addition, transfection can be mediated by a transfection agent. A skilled artisan would appreciate that the term “transfection agent” may encompass any compound that mediates incorporation of DNA in the host cell, e.g., liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

Transfection may be “stable” (i.e. integration of the foreign DNA into the host genome) or “transient” (i.e., DNA is episomally expressed in the host cells) or mRNA is electroporated into cells).

For stable transfection of mammalian cells, it is known that, depending upon the expression vector and transfection technique used, only a small fraction of cells may integrate the foreign DNA into their genome the remainder of the DNA remains episomal. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding PDX-1 or can be introduced on a separate vector. Cells stably transfected with the introduced nucleic acid can be identified by drug selection (e.g., cells that have incorporated the selectable marker gene will survive, while the other cells die). In another embodiment, the cells modulated by PDX-1 or the transfected cells are identified by the induction of expression of an endogenous reporter gene. In some embodiments, the promoter is the insulin promoter driving the expression of green fluorescent protein (GFP).

In some embodiments the PDX-1, Pax-4, MafA, NeuroD1, or Sox-9 nucleic acid is present in a viral vector. In some embodiments, the PDX-1 and NeuroD1 nucleic acids are present in the same viral vector. In another embodiment, the PDX-1 and Pax4 nucleic acids are present in the same viral vector. In another embodiment the PDX-1, Pax-4, MafA, NeuroD1, or Sox-9 nucleic acid is encapsulated in a virus. In another embodiment, the PDX-1 and NeuroD1 is encapsulated in a virus (i.e., nucleic acids encoding PDX-1 and NeuroD1 are encapsulated in the same virus particle). In another embodiment, the PDX-1 and Pax4 are encapsulated in a virus (i.e., nucleic acids encoding PDX-1 and Pax4 are encapsulated in the same virus particle). In some embodiments, the virus infects pluripotent cells of various tissue types, e.g. hematopoietic stem, cells, neuronal stem cells, hepatic stem cells or embryonic stem cells. In some embodiments, the virus is hepatotropic. A skilled artisan would appreciate that the term “hepatotropic” it is meant that the virus has the capacity to target the cells of the liver either specifically or non-specifically. In further embodiments, the virus is a modulated hepatitis virus, SV-40, or Epstein-Bar virus. In yet another embodiment, the virus is an adenovirus.

In some embodiments, 3D cell clusters are dissociated into single cells. In some embodiments, dissociating can be effectuated with any enzyme or combination of enzymes having proteolytic and/or collagenolytic activity. In some embodiments, dissociation is effectuated with trypsin, collagenase, hyaluronidase, papain, protease type XIV, pronase and/or proteinase K. In some embodiments, dissociation is effectuated with Accutase®. In some embodiments, dissociated cells are further seeded in adherent conditions.

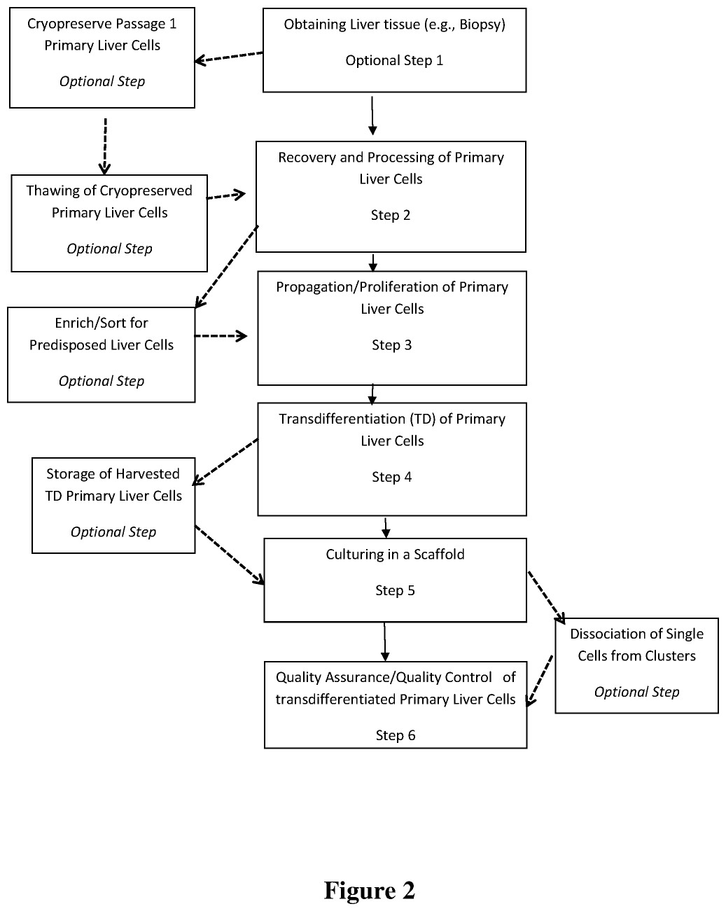

FIG. 2 describes one embodiment of a manufacturing process of human insulin producing cells, wherein the starting material comprises liver tissue. A skilled artisan would recognize that any source of non-pancreatic β-cell tissue could be used in this manufacturing process.

Embodiments for many of the steps presented in FIG. 2 are described in detail throughout this application, and will not be repeated herein, though they should be considered herein. Reference is also made to Examples 1-2, which exemplify many of these steps. In brief, the manufacturing process may be understood based on the steps presented below.

As indicated at Optional step 1: Obtaining Liver Tissue. In some embodiments, primary cells are obtained from a tissue or organ. In some embodiments, liver tissue is human liver tissue. In another embodiment, the liver tissue is obtained as part of a biopsy. In another embodiment, liver tissue is obtained by way of any surgical procedure known in the art. In another embodiment, obtaining liver tissue is performed by a skilled medical practitioner. In another embodiment, liver tissue obtained is liver tissue from a healthy individual. In a related embodiment, the healthy individual is an allogeneic donor for a patient in need of a cell-based therapy that provides processed insulin in a glucose regulated manner, for example a type I Diabetes mellitus patient or a patient suffering for pancreatitis. In another embodiment, donor Screening and Donor Testing was performed to ensure that tissue obtained from donors shows no clinical or physical evidence of or risk factors for infectious or malignant diseases were from manufacturing of AIP cells. In yet another embodiment, liver tissue is obtained from a patient in need of a cell-based therapy that provides processed insulin in a glucose regulated manner, for example a type I Diabetes mellitus patient or a patient suffering for pancreatitis. In still another embodiment, liver tissue is autologous with a patient in need of a cell-based therapy that provides processed insulin in a glucose regulated manner, for example a type I Diabetes mellitus patient or a patient suffering for pancreatitis.

As indicated at Step 2: Recovery and Processing of Primary Liver Cells. Liver tissue is processed using well know techniques in the art for recovery of adherent cells to be used in further processing. In some embodiments, liver tissue is cut into small pieces of about 1-2 mm and gently pipetted up and down in sterile buffer solution. The sample may then be incubated with collagenase to digest the tissue. Following a series of wash steps, in another embodiment, primary liver cells may be plated on pre-treated fibronectin-coated tissue culture tissue dishes. A skilled artisan would then process (passage) the cells following well-known techniques for propagation of liver cells. Briefly, cells may be grown in a propagation media and through a series of seeding and harvesting cell number is increased. Cells may be split upon reaching 80% confluence and re-plated. In another embodiment, following wash steps, primary liver cells are seeded under non-adherent conditions. In one embodiment, following wash steps, primary liver cells are attached to a scaffold.

A skilled artisan would appreciate the need for sufficient cells at, for example the 2-week time period, prior to beginning the expansion phase of the protocol (step 3). The skilled artisan would recognize that the 2-week time period is one example of a starting point for expanding cells, wherein cells may be ready for expansion be before or after this time period. In some embodiments, recovery and processing of primary cells yields at least 1×10⁵ cells after two passages of the cells. In another embodiment, recovery and processing of primary cells yields at least 1×10⁶ cells after two passages of the cells. In another embodiment, recovery and processing of primary cells yields at least 2×10⁶ cells after two passages of the cells. In another embodiment, recovery and processing of primary cells yields at least 5×10⁶ cells after two passages of the cells. In another embodiment, recovery and processing of primary cells yields at least 1×10⁷ cells after two passages of the cells. In another embodiment, recovery and processing of primary cells yields between 1×10⁵-1×10⁶ cells after two passages of the cells. In another embodiment, recovery and processing of primary cells yields between 1×10⁶-1×10⁷ cells after two passages of the cells. In another embodiment, enough starting tissue is used to ensure the recovery and processing of primary cells yields enough cells after two passages for an adequate seeding density at Step 3, large-scale expansion of the cells.

In another embodiment, early passage primary cells are cryopreserved for later use. In some embodiments, 1^{st} passage primary cells are cryopreserved for later use. In yet another embodiment, 2^{nd} passage primary cells are cryopreserved for later use.

As indicated at Step 3: Propagation/Proliferation of Primary Liver Cells. Step 3 represents the large-scale expansion phase of the manufacturing process. In some embodiments, cells propagate/proliferate on a scaffold. A skilled artisan would appreciate the need for sufficient cells at the 5-week time period, prior to beginning the transdifferentiation phase of the protocol (step 4), wherein a predetermined number of cells may be envisioned to be needed for treating a patient. In some embodiments, the predetermined number of cells needed prior to transdifferentiation comprises about 1×10⁸ primary cells. In another embodiment, the predetermined number of cells needed prior to transdifferentiation comprises about 2×10⁸ primary cells. In some embodiments, the predetermined number of cells needed prior to transdifferentiation comprises about 3×10⁸ primary cells, 4×10⁸ primary cells, 5×10⁸ primary cells, 6×10⁸ primary cells, 7×10⁸ primary cells, 8×10⁸ primary cells, 9×10⁸ primary cells, 1×10⁹ primary cells, 2×10⁹ primary cells, 3×10⁹ primary cells, 4×10⁹ primary cells, 5×10⁹ primary cells, 6×10⁹ primary cells, 7×10⁹ primary cells, 8×10⁹ primary cells, 9×10⁹ primary cells, or 1×10¹⁰ primary cells.

In some embodiments, cells are seeded on a scaffold. In some embodiments, the cell seeding density at the time of expansion comprises 1×10³-10×10³ cell/cm². In another embodiment, the cell seeding density at the time of expansion comprises 1×10³-8×10³ cell/cm². In another embodiment, the cell seeding density at the time of expansion comprises 1×10³-5×10³ cell/cm². In another embodiment, the cell seeding density at the time of expansion comprises 5×10³-10×10³ cell/cm². In another embodiment, the cell seeding density at the time of expansion comprises 10×10³-20×10³ cell/cm². In another embodiment, the cell seeding density at the time of expansion comprises 20×10³-30×10³ cell/cm². In another embodiment, the cell seeding density at the time of expansion comprises 30×10³-40×10³ cell/cm². In another embodiment, the cell seeding density at the time of expansion comprises 40×10³-50×10³ cell/cm². In another embodiment, the cell seeding density at the time of expansion comprises 50×10³-100×10³ cell/cm².

In another embodiment, the cell seeding density at the time of expansion comprises about 1×10³ cells/cm². In another embodiment, the cell seeding density at the time of expansion comprises about 2×10³ cells/cm². In another embodiment, the cell seeding density at the time of expansion comprises about 3×10³ cells/cm². In another embodiment, the cell seeding density at the time of expansion comprises about 4×10³ cells/cm². In another embodiment, the cell seeding density at the time of expansion comprises about 5×10³ cells/cm². In another embodiment, the cell seeding density at the time of expansion comprises about 6×10³ cells/cm². In another embodiment, the cell seeding density at the time of expansion comprises about 7×10³ cells/cm². In another embodiment, the cell seeding density at the time of expansion comprises about 8×10³ cells/cm². In another embodiment, the cell seeding density at the time of expansion comprises about 9×10³ cells/cm². In another embodiment, the cell seeding density at the time of expansion comprises about 10×10³ cells/cm². In another embodiment, the cell seeding density at the time of expansion comprises about 20×10³ cells/cm². In another embodiment, the cell seeding density at the time of expansion comprises about 40×10³ cells/cm². In another embodiment, the cell seeding density at the time of expansion comprises about 60×10³ cells/cm².

In another embodiment, the range for cells seeding viability at the time of expansion comprises 60-100%. In another embodiment, the range for cells seeding viability at the time of expansion comprises a viability of about 70-99%. In another embodiment, the cell seeding viability at the time of expansion comprises a viability of about 60%. In another embodiment, the cell seeding viability at the time of expansion comprises a viability of about 65%. In another embodiment, the cell seeding viability at the time of expansion comprises a viability of about 70%. In another embodiment, the cell seeding viability at the time of expansion comprises a viability of about 75%. In another embodiment, the cell seeding viability at the time of expansion comprises a viability of about 80%. In another embodiment, the cell seeding viability at the time of expansion comprises a viability of about 85%. In another embodiment, the cell seeding viability at the time of expansion comprises a viability of about 90%. In another embodiment, the cell seeding viability at the time of expansion comprises a viability of about 95%. In another embodiment, the cell seeding viability at the time of expansion comprises a viability of about 99%. In another embodiment, the cell seeding viability at the time of expansion comprises a viability of about 99.9%.

A skilled artisan would recognize variability within starting tissue material. Therefore, in another embodiment expansion occurs between weeks 2 and 6. In still another embodiment, expansion occurs between weeks 2 and 7. In another embodiment, expansion occurs between weeks 2 and 4. In yet another embodiment, expansion occurs until the needed number of primary cells has been propagated.

In some embodiments, bioreactors are used to expand and propagate primary cells prior to the transdifferentiation step. In some embodiments, cells aggregated in 3D clusters attached to a scaffold are propagated in bioreactors. Bioreactors may be used or cultivation of cells, in which conditions are suitable for high cell concentrations. In another embodiment, a bioreactor provides a closed system for expansion of cells. In another embodiment, multiple bioreactors are used in a series for cell expansion. In another embodiment, a bioreactor used in the methods disclosed herein is a single use bioreactor. In another embodiment, a bioreactor used is a multi-use bioreactor. In yet another embodiment, a bioreactor comprises a control unit for monitoring and controlling parameters of the process. In another embodiment, parameters for monitoring and controlling comprise Dissolve Oxygen (DO), pH, gases, and temperature.

In some embodiments, primary liver cells are propagated under non-adherent conditions. In some embodiments, primary liver cells are attached to a scaffold. In some embodiments, primary liver cells are propagated on a scaffold.

As indicated at Step 4: Transdifferentiation (TD) of primary Liver Cells. In some embodiments, transdifferentiation comprises any method of transdifferentiation disclosed herein. For example, transdifferentiation may comprise a “hierarchy” (1+1+1) protocol or a “2+1” protocol, as disclosed herein. In some embodiments, a “hierarchy” or 1+1+1 protocol refers to a protocol in which 3 pTFs are administered in a sequential manner and according to the order in which they're expressed during pancreatic beta cell differentiation. In some embodiment, the 3 pTFs are PDX-1, NeuroD1 and MafA. In some embodiments, “2+1” protocol refers to a transdifferentiation protocol in which 2 pTFs are administered at a first time and a third pTF is administered at a subsequent second time.

In some embodiments, the resultant cell population following transdifferentiation comprises transdifferentiated cells having a pancreatic phenotype and function. In another embodiment, the resultant cell population following transdifferentiation comprises transdifferentiated cells having a mature β-cell pancreatic phenotype and function. In another embodiment, the resultant cell population following transdifferentiation comprises transdifferentiated cells having increased insulin content. In another embodiment, the resultant cell population following transdifferentiation comprises transdifferentiated cells able to secrete processed insulin in a glucose-regulated manner In another embodiment, the resultant cell population following transdifferentiation comprises transdifferentiated cells has increased C-peptide levels.

In another embodiment, the resultant cell population following transdifferentiation comprises transdifferentiated cells having increased endogenous expression of at least one pancreatic gene marker. In another embodiment, endogenous expression is increased for at least two pancreatic gene markers. In another embodiment, endogenous expression is increased for at least three pancreatic gene markers. In another embodiment, endogenous expression is increased for at least four pancreatic gene markers. In a related embodiment, pancreatic gene markers comprise PDX-1, NeuroD1, MafA, Nkx6.1, glucagon, somatostatin and Pax4.

In some embodiments, endogenous PDX-1 expression is greater than 10² fold over non-transdifferentiated cells. In another embodiment, endogenous PDX-1 expression is greater than 10³ fold over non-transdifferentiated cells. In another embodiment, endogenous PDX-1 expression is greater than 10⁴ fold over non-transdifferentiated cells. In another embodiment, endogenous PDX-1 expression is greater than 10⁵ fold over non-transdifferentiated cells. In another embodiment, endogenous PDX-1 expression is greater than 10⁶ fold over non-transdifferentiated cells.

In another embodiment, endogenous NeuroD1 expression is greater than 10² fold over non-transdifferentiated cells. In another embodiment, endogenous NeuroD1 expression is greater than 10³ fold over non-transdifferentiated cells. In another embodiment, endogenous NeuroD1 expression is greater than 10⁴ fold over non-transdifferentiated cells. In another embodiment, endogenous NeuroD1 expression is greater than 10⁵ fold over non-transdifferentiated cells.

In another embodiment, endogenous MafA expression is greater than 10² fold over non-transdifferentiated cells. In another embodiment, endogenous MafA expression is greater than 10³ fold over non-transdifferentiated cells. In another embodiment, endogenous MafA expression is greater than 10⁴ fold over non-transdifferentiated cells. In another embodiment, endogenous MafA expression is greater than 10⁵ fold over non-transdifferentiated cells.

In another embodiment, endogenous glucagon expression is greater than 10 fold over non-transdifferentiated cells. In another embodiment, endogenous glucagon expression is greater than 10² fold over non-transdifferentiated cells. In another embodiment, endogenous glucagon expression is greater than 10³ fold over non-transdifferentiated cells.

In another embodiment, endogenous expression of PDX-1, NeuroD1, or MafA, or any combination thereof is each greater than 60% over non-transdifferentiated cells. In another embodiment, endogenous expression of PDX-1, NeuroD1, or MafA, or any combination thereof is each greater than 70% over non-transdifferentiated cells. In another embodiment, endogenous expression of PDX-1, NeuroD1, or MafA, or any combination thereof is each greater than 80% over non-transdifferentiated cells

In another embodiment, the resultant cell population following transdifferentiation comprises transdifferentiated cells having at least 60% viability. In another embodiment, the resultant cell population following transdifferentiation comprises transdifferentiated cells having at least 70% viability. In another embodiment, the resultant cell population following transdifferentiation comprises transdifferentiated cells having at least 80% viability. In another embodiment, the resultant cell population following transdifferentiation comprises transdifferentiated cells having at least 90% viability.

In some embodiments, the cells exhibiting a mature beta-cell phenotype generated by the methods described herein may repress at least one gene or the gene expression profile of the original cell. For example, a liver cell that is induced to exhibit a mature beta-cell phenotype may repress at least one liver-specific gene. One skilled in the art could readily determine the liver-specific gene expression of the original cell and the produced cells using methods known in the art, i.e. measuring the levels of mRNA or polypeptides encoded by the genes. Upon comparison, a decrease in the liver-specific gene expression would indicate that transdifferentiation has occurred.

In certain embodiments, the transdifferentiated cells disclosed herein comprise a reduction of liver phenotypic markers. In some embodiments, there is a reduction of expression of albumin, alpha-1 anti-trypsin, or a combination thereof. In another embodiment, less than 5% of the cell population expressing endogenous PDX-1 expresses albumin and alpha-1 anti-trypsin. In another embodiment, less than 10%, 9%, 8%, 7%, 6%, 4%, 3%, 2%, or 1% of the transdifferentiated cells expressing endogenous PDX-1 expresses albumin and alpha-1 anti-trypsin.

In another embodiment, transdifferentiated cells maintain a pancreatic phenotype and function for at least 6 months. In another embodiment, transdifferentiated cells maintain a pancreatic phenotype and function for at least 12 months. In another embodiment, transdifferentiated cells maintain a pancreatic phenotype and function for at least 18 months. In another embodiment, transdifferentiated cells maintain a pancreatic phenotype and function for at least 24 months. In another embodiment, transdifferentiated cells maintain a pancreatic phenotype and function for at least 36 months. In another embodiment, transdifferentiated cells maintain a pancreatic phenotype and function for at least 48 months.

In another embodiment, transdifferentiated cells maintain a pancreatic phenotype and function for at least 4 years. In another embodiment, transdifferentiated cells maintain a pancreatic phenotype and function for at least 5 years.

In some embodiments, cell number is maintained during transdifferentiation. In another embodiment, cell number decreases by less than 5% during transdifferentiation. In another embodiment, cell number decreases by less than 10% during transdifferentiation. In another embodiment, cell number decreases by less than 15% during transdifferentiation. In another embodiment, cell number decreases by less than 20% during transdifferentiation. In another embodiment, cell number decreases by less than 25% during transdifferentiation.

In some embodiments, primary liver cells are transdifferentiated under non-adherent conditions. In some embodiments, primary liver cells are seeded on a scaffold and transdifferentiated on it.

As indicated at Step 5: Culturing in a Scaffold. In some embodiments, transdifferentiated cells are seeded in a scaffold. In some embodiments, the cell seeding density comprises 1×10³-10×10³ cell/cm². In another embodiment, the cell seeding density comprises 1×10³-8×10³ cell/cm². In another embodiment, the cell seeding density comprises 1×10³-5×10³ cell/cm². In another embodiment, the cell seeding density comprises 1×10³ cell/cm². In another embodiment, the cell seeding density comprises 2×10³ cell/cm². In another embodiment, the cell seeding density comprises 3×10³ cell/cm². In another embodiment, the cell seeding density comprises 4×10³ cell/cm². In another embodiment, the cell seeding density comprises 5×10³ cell/cm². In another embodiment, the cell seeding density comprises 6×10³ cell/cm². In another embodiment, the cell seeding density comprises 7×10³ cell/cm². In another embodiment, the cell seeding density comprises 8×10³. In another embodiment, the cell seeding density comprises 9×10³ cell/cm². In another embodiment, the cell seeding density comprises 10×10³ cell/cm².

In some embodiments, the seeded cells are in contact with a medium. In some embodiments, cells are seeded at a density of 5×10³ o 10×10³ cells/ml. In some embodiments, cells are seeded at a density of 10×10³ to 20×10³ cells/ml. In some embodiments, cells are seeded at a density of 20×10³ to 30×10³ cells/ml. In some embodiments, cells are seeded at a density of 30×10³ to 40×10³ cells/ml. In some embodiments, cells are seeded at a density of 40×10³ to 50×10³ cells/ml. In some embodiments, cells are seeded at a density of 50×10³ to 60×10³ cells/ml. In some embodiments, cells are seeded at a density of 60×10³ to 70×10³ cells/ml. In some embodiments, cells are seeded at a density of 70×10³ to 80×10³ cells/ml. In some embodiments, cells are seeded at a density of 80×10³ to 90×10³ cells/ml. In some embodiments, cells are seeded at a density of 90×10³ to 100×10³ cells/ml. In some embodiments, cells are seeded at a density of 100×10³ to 200×10³ cells/ml. In some embodiments, cells are seeded at a density of 200×10³ to 500×10³ cells/ml. In some embodiments, cells are seeded at a density of over 500×10³ cells/ml.

A skilled artisan would appreciate that the size of a 3D cell cluster is determined, amongst others, by cell seeding and cell culture conditions. In some embodiments, the size of a 3D cell cluster is determined by the number of cells seeded. In some embodiments, the size of a 3D cell cluster is determined by the well area. In some embodiments, the size of a 3D cell cluster is determined by the well volume. In some embodiments, the size of a 3D cell cluster is determined by the well shape. In some embodiments, the size of a 3D cell cluster is determined by the cell/area concentration. In some embodiments, the size of a 3D cell cluster is determined by the cell/volume concentration.

In some embodiments, a predetermined number of cells is seeded in a well having a predetermined area. In some embodiments, a predetermined number of cells is seeded in a well having a predetermined volume. A skilled artisan would appreciate that 3D cell cluster size can be optimized by seeding different cell numbers in wells of different volumes, determining average 3D cell cluster size by known methods, and selecting the cell number and well volume by which the desired size is obtained.

A skilled artisan would appreciate that uniform aggregates of a predetermined size can be generated by using microwells, as described in Urdin et al. PLoS One (2008) 3(2): e1565. In short, cells are seeded in a well or a plate comprising a textured surface consisting of numerous collecting volumes, or microwells, at the base of the plate. Said microwells might have angled collecting surfaces sloped towards a common collecting point. In some embodiments, cells are loaded into plates prepared as above in 100 or 25 μL volume for 96- and 384-well plates, respectively. Plates are then centrifuged for 5 minutes at 200×g, and then incubated. In some embodiments, well contents are recovered via inverted centrifugation for 1 minute at 50×g. In some embodiments, well contents are recovered by pipetting with large bore “genomic” pipette tips (Molecular Bioproducts, cat #3531). Resulting aggregates can be dispensed over an inverted filter unit to eliminate unincorporated cells, debris, or small cell clusters.

A skilled artisan would appreciate that a desired 3D cell cluster size might be obtained by modifying variables such as the number of cells seeded and/or the microwell volume. In some embodiments, the terms “microwells”, “wells”, “collecting volumes”, “chambers”, and “microchambers” are used herein interchangeably, having all the same qualities and meanings.

In some embodiments, a microwell has a volume smaller than 20 μm³. In some embodiments, a microwell has a volume ranging from about 20 μm³ to about 50 μm³. In some embodiments, a microwell has a volume ranging from about 50 μm³ to about 100 μm³. In some embodiments, a microwell has a volume ranging from about 100 μm³ to about 250 μm³. In some embodiments, a microwell has a volume ranging from about 250 μm³ to about 500 μm³. In some embodiments, a microwell has a volume ranging from about 500 μm³ to about 750 μm³. In some embodiments, a microwell has a volume ranging from about 750 μm³ to about 1000 μm³. In some embodiments, a microwell has a volume larger than 1000 μm³. In some embodiments, a microwell has a volume of 400 μm³.

In some embodiments, an average of less than 10 cells are seeded in each microwell. In some embodiments, an average of between 10 and 50 cells are seeded in each microwell. In some embodiments, an average of between 50 and 250 cells are seeded in each microwell. In some embodiments, an average of between 250 and 500 cells are seeded in each microwell. In some embodiments, an average of between 500 and 1000 cells are seeded in each microwell. In some embodiments, an average of more than 1000 cells are seeded in each microwell. In some embodiments, an average of about 150 cells are seeded in each microwell.

In some embodiments, the density of transdifferentiated cells on the scaffold at the end of the production process is about 1×10³-1×10⁵ cells/cm². In another embodiment, the density of transdifferentiated cells on the scaffold at the end of the production process is about 1×10⁴-5×10⁴ cells/cm². In another embodiment, the density of transdifferentiated cells on the scaffold at the end of the production process is about 1×10⁴-4 ×10⁴ cells/cm². In another embodiment, the density of transdifferentiated cells on the scaffold at the end of the production process is about 1×10³ cells/cm². In another embodiment, the density of transdifferentiated cells on the scaffold at the end of the production process is about 2×10³ cells/cm². In another embodiment, the density of transdifferentiated cells on the scaffold at the end of the production process is about 3×10³ cells/cm². In another embodiment, the density of transdifferentiated cells on the scaffold at the end of the production process is about 4×10³ cells/cm². In another embodiment, the density of transdifferentiated cells on the scaffold at the end of the production process is about 5×10³ cells/cm². In another embodiment, the density of transdifferentiated cells on the scaffold at the end of the production process is about 6×10³ cells/cm². In another embodiment, the density of transdifferentiated cells on the scaffold at the end of the production process is about 7×10³ cells/cm². In another embodiment, the density of transdifferentiated cells on the scaffold at the end of the production process is about 8×10³ cells/cm². In another embodiment, the density of transdifferentiated cells on the scaffold at the end of the production process is about 9×10³ cells/cm². In another embodiment, the density of transdifferentiated cells on the scaffold at the end of the production process is about 1×10⁴ cells/cm². In another embodiment, the density of transdifferentiated cells on the scaffold at the end of the production process is about 2×10⁴ cells/cm². In another embodiment, the density of transdifferentiated cells on the scaffold at the end of the production process is about 3×10⁴cells/cm². In another embodiment, the density of transdifferentiated cells on the scaffold at the end of the production process is about 4×10⁴ cells/cm². In another embodiment, the density of transdifferentiated cells on the scaffold at the end of the production process is about 5×10⁴cells/cm². In another embodiment, the density of transdifferentiated cells on the scaffold at the end of the production process is about 6×10⁴ cells/cm². In another embodiment, the density of transdifferentiated cells on the scaffold at the end of the production process is about 7×10⁴cells/cm². In another embodiment, the density of transdifferentiated cells on the scaffold at the end of the production process is about 8×10⁴ cells/cm². In another embodiment, the density of transdifferentiated cells on the scaffold at the end of the production process is about 9×10⁴ cells/cm².

In another embodiment, the range for cell viability at the end of the production process comprises 50-100%. In another embodiment, the range for cell viability at the end of the production process comprises 60-100%. In another embodiment, the range for cell viability at the end of the production process comprises 50-90%. In another embodiment, the range for cell viability at the end of the production process comprises a viability of about 60-99%. In another embodiment, the range for cell viability at the end of the production process comprises a viability of about 60-90%. In another embodiment, the cell viability at the end of the production process comprises a viability of about 60%. In another embodiment, the cell viability at the end of the production process comprises a viability of about 65%. In another embodiment, the cell viability at the end of the production process comprises a viability of about 70%. In another embodiment, the cell viability at the end of the production process comprises a viability of about 75%. In another embodiment, the cell viability at the end of the production process comprises a viability of about 80%. In another embodiment, the cell viability at the end of the production process comprises a viability of about 85%. In another embodiment, the cell viability at the end of the production process comprises a viability of about 90%. In another embodiment, the cell viability at the end of the production process comprises a viability of about 95%. In another embodiment, the cell viability at the end of the production process comprises a viability of about 99%. In another embodiment, the cell viability at the end of the production process comprises a viability of about 99.9%.

In another embodiment, transdifferentiated primary liver cells comprising human insulin producing cells are stored for use in a cell-based therapy at a later date. In another embodiment, storage comprises cryopreserving the cells.

In some embodiments, harvested 3D cell clusters are dissociated into single cells. Cells can be dissociated by using any enzyme or combination of enzymes having proteolytic activity or collagenolytic activity. In some embodiments, cells are dissociated by using trypsin. In some embodiments, cells are dissociated by using Accuttase®. In some embodiments, dissociated cells are seeded under attachment conditions.

In some embodiments, 3D cell clusters having one or more desired features are separated. In some embodiments, said desired features are selected from a group comprising: 3D cluster size, 3D cluster volume, 3D cluster number of cells, 3Dc luster cells surface markers. A skilled artisan would appreciate that methods for separating 3D clusters according to these desired features are well known in the art.

In some embodiments, cell clusters are separated by their size. In some embodiments, said separation by size comprises a step of filtration. Separation by filtration comprises seeding cell clusters on a filter with pores of a predetermined size, wherein clusters smaller than the pores pass through it, while clusters larger than the pores are retained. In some embodiments, said separation by size comprises a step of centrifugation. In some embodiments, said separation by size comprises a step of sedimentation.

In some embodiments, 3D cell clusters are separated by using a filter with pores ranging from 5 μm to 10 μm. In some embodiments, 3D cell clusters are separated by using a filter with pores ranging from 10 μm to 25 μm. In some embodiments, 3D cell clusters are separated by using a filter with pores ranging from 25 μm to 50 μm. In some embodiments, 3D cell clusters are separated by using a filter with pores ranging from 50 μm to 75 μm. In some embodiments, 3D cell clusters are separated by using a filter with pores ranging from 75 μm to 100 μm. In some embodiments, 3D cell clusters are separated by using a filter with pores ranging from 100 μm to 250 μm. In some embodiments, 3D cell clusters are separated by using a filter with pores ranging from 250 μm to 500 μm. In some embodiments, 3D cell clusters are separated by using a filter with pores ranging from 500 μm to 750 μm. In some embodiments, 3D cell clusters are separated by using a filter with pores ranging from 750 μm to 1000 μm. In some embodiments, 3D cell clusters are separated by using a filter with pores larger than 1000 μm.

As indicated at Step 6: Quality Analysis/Quality Control. Before any use of transdifferentiated cells in a cell-based therapy, the transdifferentiated cells must undergo a quality analysis/quality control assessment. FACS analysis and/or RT-PCR may be used to accurately determine membrane markers and gene expression. Further, analytical methodologies for insulin secretion are well known in the art including ELISA, MSD, ELISpot, HPLC, RP-HPLC. In some embodiments, insulin secretion testing is at low glucose concentrations (about 2 mM) in comparison to high glucose concentrations (about 17.5 mM).

Methods of Treating a Pancreatic Disorder

Disclosed herein are methods for treating a pancreatic disease or disorder in a subject, the methods comprising providing tridimensional (3D) cell clusters comprising transdifferentiated cells having a mature pancreatic beta cell phenotype, wherein at least a subset of the cells are attached to a scaffold. In some embodiments, treating a pancreatic disease or disorder comprises preventing or delaying the onset or alleviating a symptom of the disease or disorder.

In some embodiments, the 3D cell cluster is administered intradermally In some embodiments, the 3D cell cluster is administered intraperitoneally. In some embodiments, the 3D cell cluster is administered surgically. In some embodiments, the 3D cell cluster is implanted under the left kidney capsule. In some embodiments, the 3D cell cluster is implanted in the hepatic portal vein. In some embodiments, the 3D cell cluster is implanted in the peritoneal cavity. In some embodiments, the 3D cell cluster is implanted in the omental punch. In some embodiments, the 3D cell cluster is implanted in the subcutaneous space. In some embodiments, the 3D cell cluster is administered in any combination of different routes.

A skilled artisan would appreciate that alternative sites for transplantation possess some characteristics that can make them advantageous over the hepatic portal vein, which is limited by low oxygen tension, as well as by potential inflammatory responses that can impair engraftment leading to significant losses to the implant. Table 1 describes some of the main advantages and disadvantages of the peritoneal cavity, the omental punch, and the subcutaneous space as sites for transplanting 3D cell cluster comprising transdifferentiated cells are attached to a scaffold.

TABLE 1
Comparison of different sites for transplantation

| Site for islet | | |
|---|---|---|
| transplantation | Advantages | Disadvantages |
| Peritoneal | Minimally invasive | Lack of re-innervation |
| cavity | laparoscopic procedure | of the graft |
| | Allows transplantation | Transplanted scaffolds may |
| | of large islet mass | clump |
| | | Difficult to locate all |
| | | scaffolds for harvesting |
| Omental pouch | Exclusive portal drainage | More complex |
| | High vascular density | transplantation |
| | Good | procedure which |
| | neoangiogenesisAccepting | does not |
| | unpurified islets | allow repeated |
| | Allowing large islet/ | transplantations |
| | IPC mass | |
| Subcutaneous | Easy accessibility | Poor blood supply |
| space | (minimally invasive | |
| | transplant procedure | |
| | and biopsies) | |
| | Allows transplantation | |
| | of large islet/IPC mass | |

In some embodiments, the pancreatic disorder is a degenerative pancreatic disorder. The methods disclosed herein are particularly useful for those pancreatic disorders that are caused by or result in a loss of pancreatic cells, e.g., islet beta cells, or a loss in pancreatic cell function. The subject is, in some embodiments, a mammal The mammal can be, e.g., a human, non-human primate, mouse, rat, dog, cat, horse, or cow.

Common degenerative pancreatic disorders include, but are not limited to: diabetes (e.g., type I, type II, or gestational) and pancreatic cancer. Other pancreatic disorders or pancreas-related disorders that may be treated by using the methods disclosed herein are, for example, hyperglycemia, pancreatitis, pancreatic pseudocysts, pancreatic trauma caused by injury, type 3 diabetes or a complication of pancreatectomy. Additionally, individuals whom have had a pancreatectomy are also suitable to treatment by the disclosed methods

In some embodiments, disclosed herein is a method for treating a pancreatic disease or disorder in a subject, the method comprising administering a 3D cell cluster comprising transdifferentiated mammalian non-pancreatic beta insulin producing cells and a scaffold. In some embodiments, disclosed herein is a method for treating type I diabetes in a subject, the method comprising administering a 3D cell cluster comprising transdifferentiated mammalian non-pancreatic beta insulin producing cells and a scaffold. In some embodiments, disclosed herein is a method for treating type II diabetes in a subject, the method comprising administering a 3D cell cluster comprising transdifferentiated mammalian non-pancreatic beta insulin producing cells and a scaffold. In some embodiments, disclosed herein is a method for treating gestational diabetes in a subject, the method comprising administering a 3D cell cluster comprising transdifferentiated mammalian non-pancreatic beta insulin producing cells and a scaffold.

In some embodiments, disclosed herein is a method for treating pancreatic cancer in a subject, the method comprising administering a 3D cell cluster comprising transdifferentiated mammalian non-pancreatic beta insulin producing cells and a scaffold. In some embodiments, disclosed herein is a method for treating hyperglycemia in a subject, the method comprising administering a 3D cell cluster comprising transdifferentiated mammalian non-pancreatic beta insulin producing cells and a scaffold. In some embodiments, disclosed herein is a method for treating pancreatitis in a subject, the method comprising administering a 3D cell cluster comprising transdifferentiated mammalian non-pancreatic beta insulin producing cells and a scaffold. In some embodiments, disclosed herein is a method for treating pancreatic pseudocysts in a subject, the method comprising administering a 3D cell cluster comprising transdifferentiated mammalian non-pancreatic beta insulin producing cells and a scaffold. In some embodiments, disclosed herein is a method for treating pancreatic trauma caused by injury in a subject, the method comprising administering a 3D cell cluster comprising transdifferentiated mammalian non-pancreatic beta insulin producing cells and a scaffold. In some embodiments, disclosed herein is a method for treating a disease caused by pancreatectomy in a subject, the method comprising administering a 3D cell cluster comprising transdifferentiated mammalian non-pancreatic beta insulin producing cells and a scaffold.

Diabetes is a metabolic disorder found in three forms: type 1, type 2 and gestational. Type 1, or IDDM, is an autoimmune disease; the immune system destroys the pancreas' insulin-producing beta cells, reducing or eliminating the pancreas' ability to produce insulin Type 1 diabetes patients must take daily insulin supplements to sustain life. Symptoms typically develop quickly and include increased thirst and urination, chronic hunger, weight loss, blurred vision and fatigue. Type 2 diabetes is the most common, found in 90 percent to 95 percent of diabetes sufferers. It is associated with older age, obesity, family history, previous gestational diabetes, physical inactivity and ethnicity. Gestational diabetes occurs only in pregnancy. Women who develop gestational diabetes have a 20 percent to 50 percent chance of developing type 2 diabetes within five to 10 years.

A subject suffering from or at risk of developing diabetes is identified by methods known in the art such as determining blood glucose levels. For example, a blood glucose value above 140 mg/dL on at least two occasions after an overnight fast means a person has diabetes. A person not suffering from or at risk of developing diabetes is characterized as having fasting sugar levels between 70-110 mg/dL.

Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia, insulin deficiency or resistance. Diabetic or pre-diabetic patients to which the compounds are administered are identified using diagnostic methods know in the art.

Hyperglycemia is a pancreas-related disorder in which an excessive amount of glucose circulates in the blood plasma. This is generally a glucose level higher than (200 mg/dl). A subject with hyperglycemia may or may not have diabetes.

Pancreatic cancer is the fourth most common cancer in the U.S., mainly occurs in people over the age of 60, and has the lowest five-year survival rate of any cancer. Adenocarcinoma, the most common type of pancreatic cancer, occurs in the lining of the pancreatic duct; cystadenocarcinoma and acinar cell carcinoma are rarer. However, benign tumors also grow within the pancreas; these include insulinoma—a tumor that secretes insulin, gastrinoma—which secretes higher-than-normal levels of gastrin, and glucagonoma—a tumor that secretes glucagon.

Pancreatic cancer has no known causes, but several risks, including diabetes, cigarette smoking and chronic pancreatitis. Symptoms may include upper abdominal pain, poor appetite, jaundice, weight loss, indigestion, nausea or vomiting, diarrhea, fatigue, itching or enlarged abdominal organs. Diagnosis is made using ultrasound, computed tomography scan, magnetic resonance imaging, ERCP, percutaneous transhepatic cholangiography, pancreas biopsy or blood tests. Treatment may involve surgery, radiation therapy or chemotherapy, medication for pain or itching, oral enzymes preparations or insulin treatment.

Pancreatitis is the inflammation and autodigestion of the pancreas. In autodigestion, the pancreas is destroyed by its own enzymes, which cause inflammation. Acute pancreatitis typically involves only a single incidence, after which the pancreas will return to normal. Chronic pancreatitis, however, involves permanent damage to the pancreas and pancreatic function and can lead to fibrosis. Alternately, it may resolve after several attacks. Pancreatitis is most frequently caused by gallstones blocking the pancreatic duct or by alcohol abuse, which can cause the small pancreatic ductules to be blocked. Other causes include abdominal trauma or surgery, infections, kidney failure, lupus, cystic fibrosis, a tumor or a scorpion's venomous sting.

Symptoms frequently associated with pancreatitis include abdominal pain, possibly radiating to the back or chest, nausea or vomiting, rapid pulse, fever, upper abdominal swelling, ascites, lowered blood pressure or mild jaundice. Symptoms may be attributed to other maladies before being identified as associated with pancreatitis.

It should be understood that the disclosure presented herein is not limited to the particular methodologies, protocols and reagents, and examples described herein. The terminology and examples used herein is for the purpose of describing particular embodiments only, for the intent and purpose of providing guidance to the skilled artisan, and is not intended to limit the scope of the disclosure presented herein.

### EXAMPLES

### Example 1: General Methods

Human liver cells: Adult human liver tissues were obtained from individuals 3-23 years old or older with the approval from the Committee of Clinical Investigations (Institutional Review Board). The isolation of human liver cells was performed as described (Sapir et al, (2005) Proc Natl Acad Sci USA 102: 7964-7969; Meivar-Levy et al, (2007) Hepatology 46: 898-905). Liver cells were cultured in Dulbecco's minimal essential medium (DMEM) (1 g/1 of glucose) supplemented with 10% fetal calf serum (FCS), 100 units/ml penicillin; 100 ng/ml streptomycin; 250 ng/ml amphotericin B (Biological Industries, Israel) at 37° C. in a humidified atmosphere of 5% CO₂ and 95% air.

Viral infection and transdifferentiation: The adenoviruses used in this study were as follows: The vectors used were Ad-CMV-Pdx-1, Ad-CMV-MafA, and Ad-CMV-NeuroD1 (WO2016108237A1). The viral particles were generated by standard protocols (He et al, (1998) Proc Natl Acad Sci USA 95: 2509-2514). The MOIs were: Ad-CMV-Pdx-1 (1000 MOI), Ad-CMV-MafA (50 MOI) and Ad-NeuroD1 (250 MOI) unless specified otherwise in an Example or Figure. Viruses were manufactured either by OD260 Inc. (ID, USA) or by Pall Inc. (USA). Cells were infected on day 1 with Ad-CMV-Pdx-1 and Ad-NeuroD1 and seeded on standard plates in TM (see below). Alternatively, cells can be infected with a single adenoviral vector encoding both PDX-1 and NeuroD1. On day 3 cells were harvested, infected with Ad-CMV-MafA and seeded under adherent or non-adherent conditions. Cells were harvested at days 6 or 7.

The cell culture media used in the experiments included: (1) transdifferentiation medium (TM, DMEM 1 g/1 of glucose supplemented with 10% fetal calf serum, 100 units/ml penicillin; 100 ng/ml streptomycin; 250 ng/ml amphotericin B, 10 mM nicotinamide (Sigma, Israel), 20 ng/ml EGF (Cytolab, Israel), 5 nM Ex4; (2) Serum free medium (SFM) consisting of DMEM 1 g/1 of glucose supplemented with 100 units/ml penicillin; 100 ng/ml streptomycin; 250 ng/ml amphotericin B 1% ITS (vol/vol), 10 mM NIC, 20 ng/ml EGF, 5 nM Ex4; and (3) “CMRL+B27” consisting of CMRL medium supplemented with100 units/ml penicillin; 100 ng/ml streptomycin; 250 ng/ml amphotericin B 2% B27 vol/vol, 10 gr/L albumin, 1% ITS, 10 mM NIC, 20 ng/ml EGF, 5nM Ex4.

Cell culture in non-adherent conditions: Three different method were used for generating three-dimensional (3D) cell clusters of transdifferentiated cells. In all methods, primary liver cells were transdifferentiated as described above in this section. However, the methods differed in the cell incubation conditions from day 3 onward.

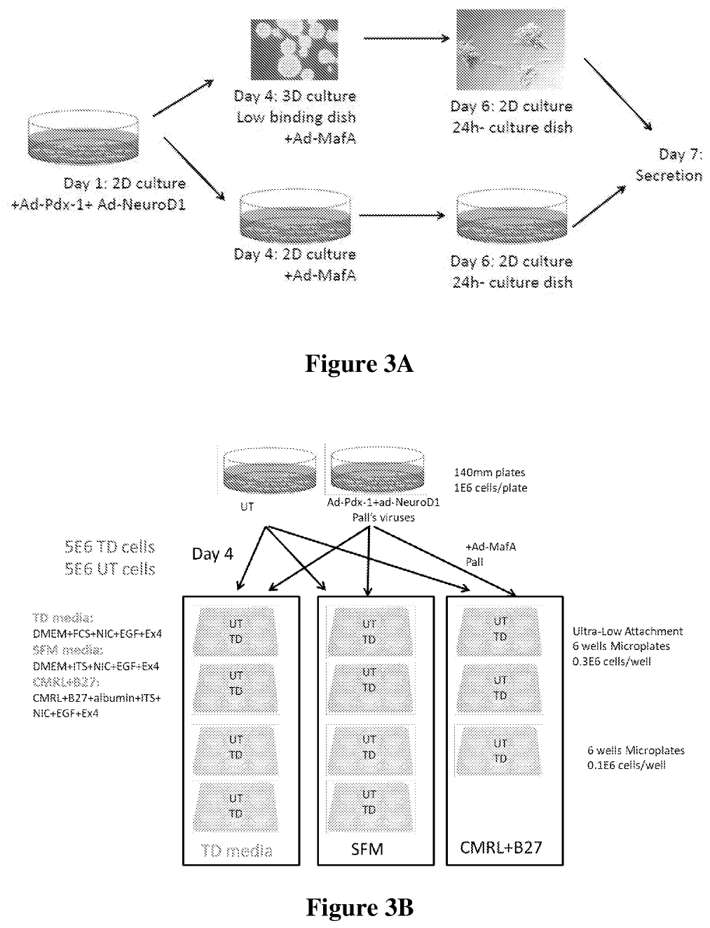

In the first method, following MafA infection on day 3, cells were seeded in low binding 6 well plates. Different cell culture media was used in different experiments, as detailed below in the Experiments section. For some experiments, cells were harvested on day 6 for RNA extraction or for GSIS studies. Afterwards, aggregates were transferred to regular culture 6 well plate. Aggregates were allowed to adhere to the plate surface for 24 h before preforming the GSIS analysis (FIG. 3A).

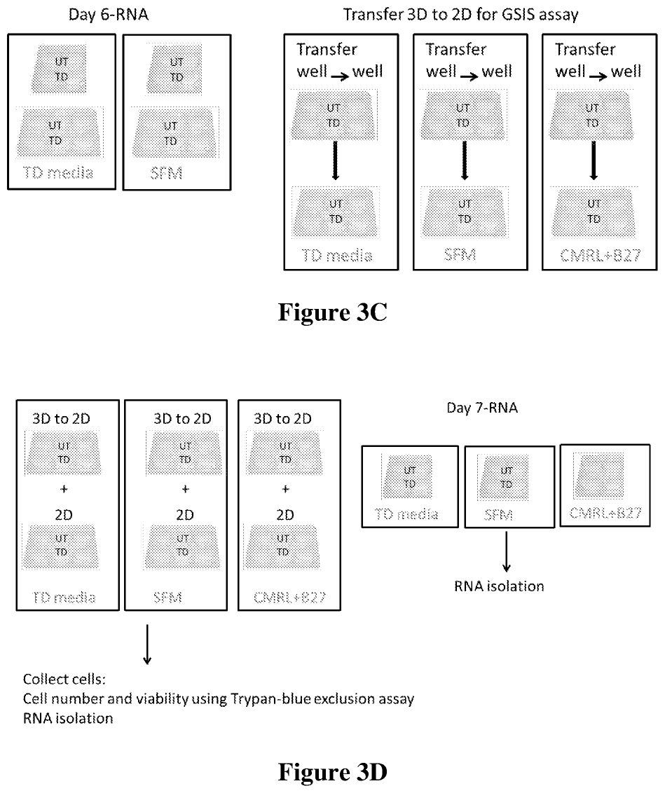

In the second method, following MafA infection on day 3, cells were seeded in low binding T-flasks. For some experiments, cells were harvested on day 6 for RNA extraction and for GSIS studies. Afterwards, aggregates were transferred into 12 μm Millicel® cell inserts (Millipore), according to manufacturer's instructions. These inserts have a 12 μm-filter bottom, which separates between large aggregates and single cells that pass freely through the filter. GSIS analysis was performed in the cell inserts (FIG. 4)

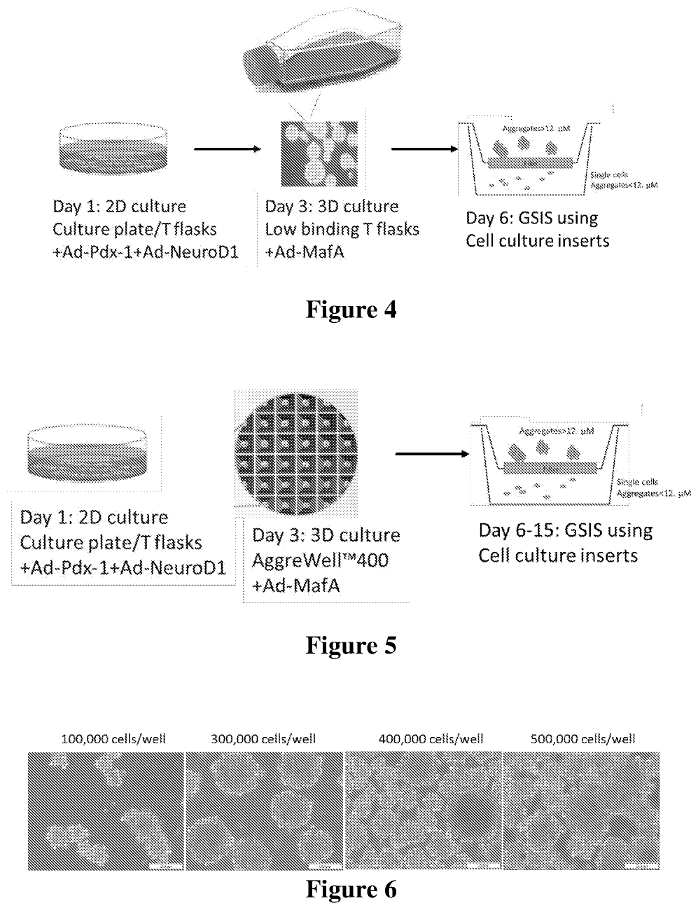

In the third method, following MafA infection on day 3, cells were seeded in AggreWell™ 400 (S rEMCELL Technologies) plates according to manufacturer's instructions. Cells were cultured in AggreWell™ plates until day 15 (FIG. 5). Cells were collected for RNA using 40 μm EASYstrainer (deGrout), or transferred into 12 μm Millicel® cell inserts (Millipore) for GSIS assay.

RNA isolation, RT and RT-PCR reactions: Total RNA was isolated and cDNA was prepared and amplified as described previously (Ber et al, (2003) J Biol Chem 278: 31950-31957; Sapir et al, (2005) ibid). Quantitative real-time-PCR was performed using ABI Step one plus sequence Detection system (Applied Biosystems, CA, USA) as described previously (Sapir et al, (2005) ibid; Meivar-Levy et al, (2007) ibid; Aviv et al, (2009) J Biol Chem 284: 33509-33520).

C-peptide and insulin secretion detection: C-peptide and insulin secretions were measured by static incubations of cultured cells 6 or 7 days following the initial exposure to the viral treatment, as described (Sapir et al, (2005) ibid; Meivar-Levy et al, (2007) ibid; Aviv et al, (2009) ibid). Glucose-stimulated insulin secretion (GSIS) was measured at 2 mM (low) and 17.5 mM (high) glucose, the latter was determined by dose-dependent analyses to maximally induce insulin secretion from transdifferentiated liver cells without having adverse effects (Sapir et al, (2005) ibid; Meivar-Levy et al, (2007) ibid; Aviv et al, (2009) ibid). C-peptide secretion was detected by radioimmunoassay using the human C-peptide radioimmunoassay kit (Linco Research, St. Charles, Mo.; <4% cross-reactivity to human proinsulin), or by ELISA using human ultra-sensitive ELISA kit (Mercodia, Uppsala, Sweden; <5% cross-reactivity to human proinsulin. Insulin secretion was detected by radioimmunoassay using human insulin radioimmunoassay kit (DPC, Angeles, Calif.; 32% cross-reactivity to human proinsulin). Cells grown in non-adherent conditions were transferred to adherent 6 wells plates prior to the assay.

Accutase® and trypsin treatment: Cells were washed with PBS then Accutase® Cell Detachment Solution (Merck Millipore) or trypsin was added (0.5 ml) for 20 min at 37° C. according to manufacture protocol

Cell Viability: Cell viability was assessed by the Trypan Blue Exclusion Assay (Sapir et al, (2005) ibid; Meivar-Levy et al, (2007) ibid).

Statistical Analysis: Statistical analyses were performed with a 2-sample Student's t-test assuming unequal variances.

### Example 2: Formation of Three-Dimensional (3D) Clusters of Untreated and Transdifferentiated Liver Cells

Objective: To determine the conditions for three-dimensional (3D) cluster formation. To compare the phenotype of insulin producing cells (IPCs) grown as 3D clusters with the phenotype of IPCs grown as monolayers.

Study Design and Methods: 3×10⁶ primary adult liver human cells were obtained from 2 different donors and transdifferentiated according to the methods described in Example 1. Following infection, cells were seeded at different concentrations (100,000-500,000 cells/well) in either standard adherent 6 wells microplates or in Corning ultra-low attachment 6 wells microplates (Sigma, Israel, Cat #: CLS3471) in serum free medium (SFM). On day 6 cells were harvested. Non-infected control cells were cultured in similar conditions.

Both Non-Infected and Transdifferentiated Cells Grown Under Non-Adherent Conditions form 3D Cell Clusters

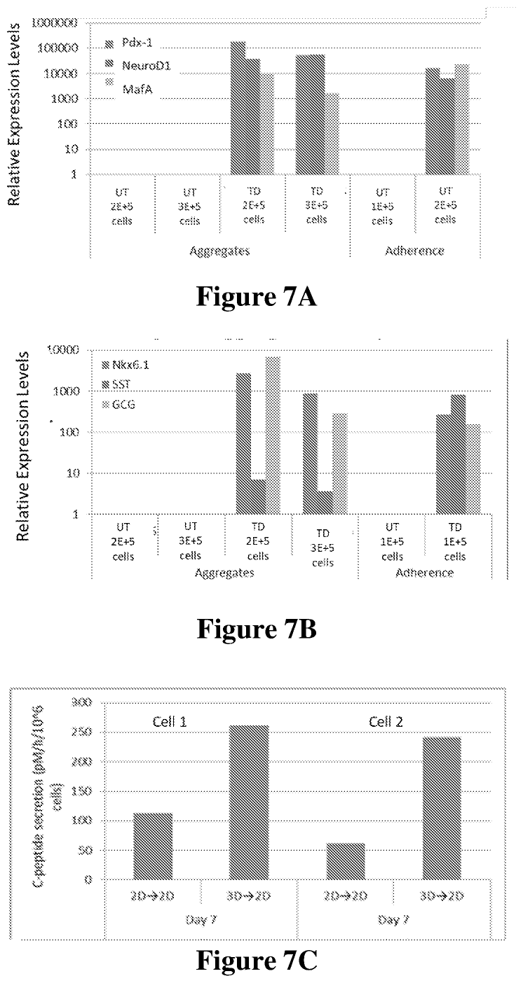

Visual observation of transdifferentiated cells cultured in ultra-low attachment 6 well plates, revealed they formed 3D clusters 4-6 h after seeding. The diameter of the clusters was correlated with the initial number of cells seeded; 100 μm diameter clusters were observed in wells seeded with 100,000 cells, and 400 μm diameter clusters were observed in wells seeded with 500,000 cells (FIG. 6). A dark core was observed in the center of 3D cell clusters bigger than 200 μm diameter, and was interpreted as a dramatic decrease in cell concentration and viability that could be due to lack of oxygen in internal regions of the cluster.

Culture of Transdifferentiated Cells under Non-Adherent Conditions Promote Mature Pancreatic 13 Cell Phenotype

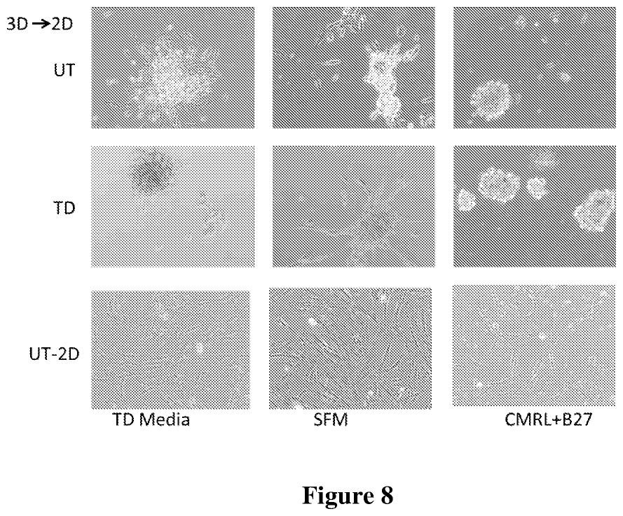

Expression levels of ectopically expressed PDX-1, NeuroD1 and MafA, as well as of endogenous mature pancreatic β cell markers NKX6.1, SST and GCG were analyzed by RT-PCR. Cells grown under non-adherent conditions expressed ectopic pTFs in similar levels to cells grown under adherent conditions (FIG. 7A). 3D clustered showed increased levels of NKX6.1, SST and GCG, suggesting that non-adherent conditions promoted a mature pancreatic β cell phenotype (FIG. 7B).

Next, glucose regulated C-peptide analysis was compared between cells grown in adherent and non-adherent conditions. Cells cultured in non-adherent conditions showed increased glucose regulated C-peptide secretion when compared with control cells cultured under adherent culture conditions (FIG. 7C).

### Example 3: Optimization of Non-Adherent Culture Methods

Objective: To determine optimal conditions for generation of three-dimensional (3D) clusters of transdifferentiated insulin producing cells (IPCs).

Study Design and Methods: 3×10⁶ primary adult liver human cells from 2 different donors were transdifferentiated according to the methods described above. Following infection, cells were seeded either in adherent 6 wells microplates or in Corning ultra-low attachment 6 wells microplates. Non-infected control cells were seeded under similar conditions. For each type of culture plate, cells were incubated either in a) TM, b) SFM, or c) CMRL+B27. On day 6 cells were harvested. Part of the cells were used for RNA extraction, and part were transferred to adherent 6 wells plates for GSIS assay. On day 7, cell number and viability were assessed, GSIS was assayed and cells' RNA extracted. A general overview of the culture method is illustrated in FIG. 3A. The conditions tested in the current experiment are illustrated in FIGS. 3B-D.

Three Dimensional (3D) Cell Clusters Maintain their Morphology when Re-Seeded under Adherent Conditions

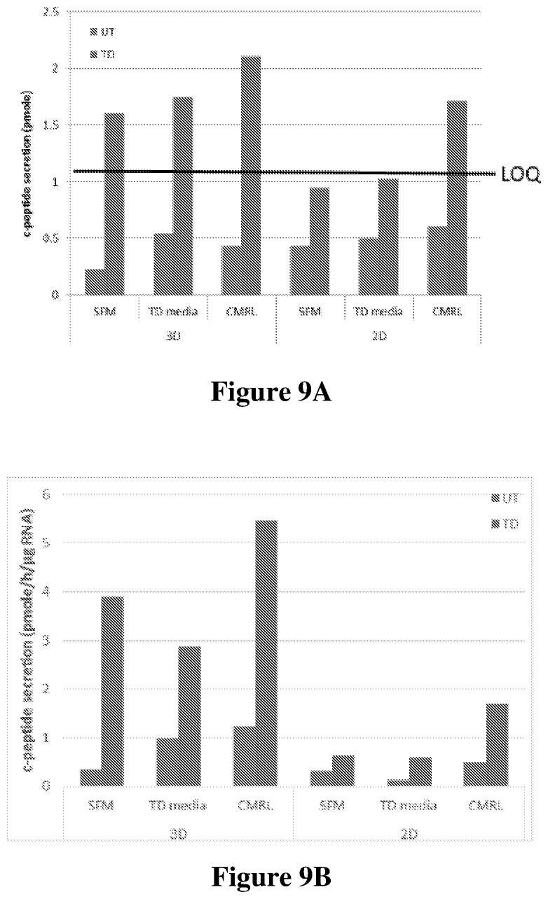

Transdifferentiated and untreated cells were grown in non-adherent conditions from day 4 to day 6 of the experiment and were then re-seeded in adherent 6 wells plates in TM, SFM or CMRL+B27 medium. Visual observation revealed that cell clusters were maintained after re-seeding (FIG. 8).

### Adherence Conditions Affect Transdifferentiation Efficacy Without Regard of the Cell Medium Used

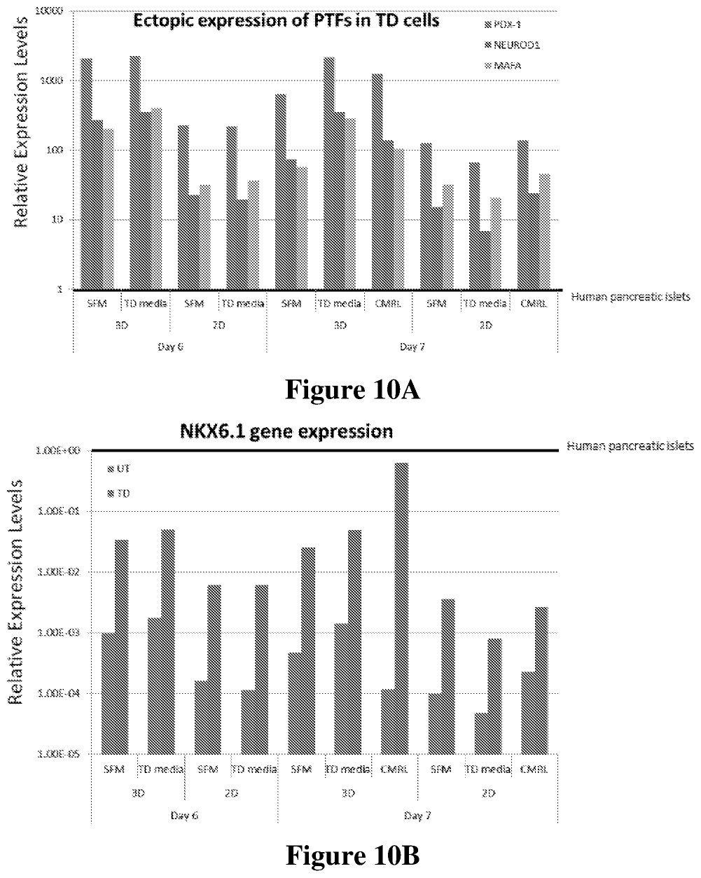

On day 7, glucose regulated C-peptide secretion was compared between cells cultured under adherent and non-adherent conditions, in TM, SFM and CMRL+B27 media. Cells grown in non-adherent conditions showed improved glucose regulated C-peptide secretion compared to cells grown on adherent conditions. The improvement was found in cells cultured in TM, SFM and CMRL+B27 media (FIGS. 9A and 9B). No significant differences were found between cells cultured in different media.

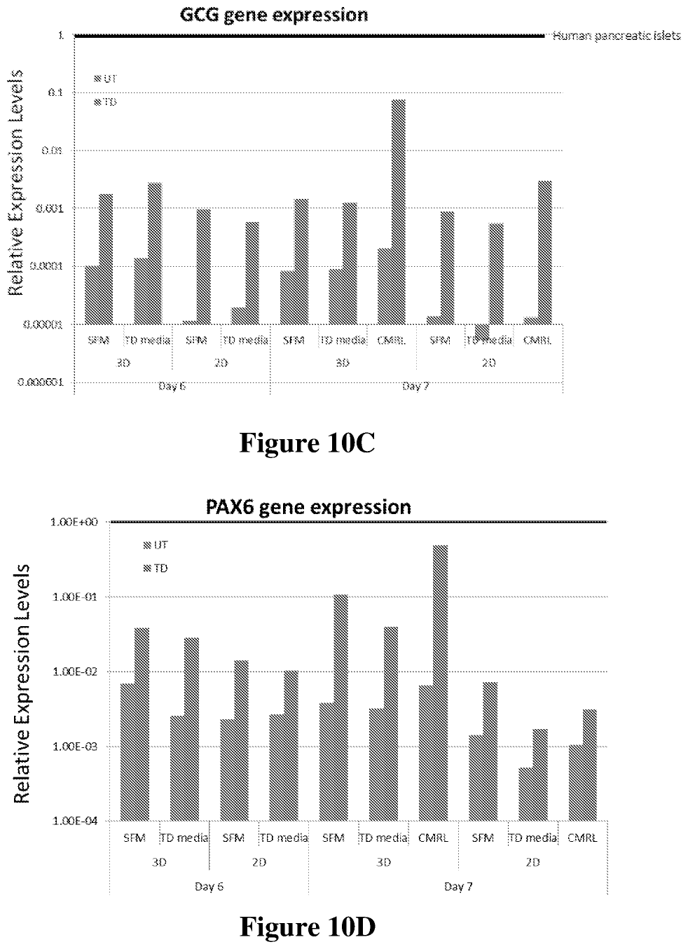

mRNA expression levels of Nkx6.1, GCG and PAX6, as well as of ectopically expressed PDX-1, NeuroD1 and MafA, were measured in cells cultured under adherent and non-adherent conditions in TM, SFM and CMRL+B27 media on days 6 and 7. Expression levels in human pancreas was used as baseline. Expression levels of ectopic genes were higher in cells grown in non-adherent conditions compared to cells grown under adherent conditions. This increase was found in all media used and both on days 6 and 7 (FIG. 10A). Assuming the infection rates in all conditions were similar, this increased expression could be explained by the lower proliferation of cells observed under non-adherent conditions, that imply a higher proportion of infected cells. Expression levels of Nkx6.1, GCG and PAX6 were higher in cells grown in non-adherent conditions compared to cells grown under adherent conditions. This increase was found in all media used and both on days 6 and 7 (FIGS. 10B, 10C and 10D).

### Example 4: Optimization of Non-Adherent Culture Methods

Objectives: 1. To optimize methods for generating clusters of transdifferentiated cells in ultra-low attachment conditions; 2. To develop methods for working in 6 wells plates thus allowing small scale cultures; 3. To develop methods for GSIS assays suitable to cell 3D cell clusters; 4. To develop methods for separating 3D clusters into single cells; and 5. To develop methods for re-seeding 3D clusters under adherent conditions.

Study Design and Methods: 3×10⁶ primary adult liver cells were transdifferentiated according to the methods described in Example 1. Following infection with MafA, cells were seeded either in a) 6 wells microplates, b) ultra-low attachment 6 wells microplates, or c) ultra-low attachment 75T flasks Corning (Sigma, Israel, Cat #: CLS3814) according to the conditions shown in Table 2. Subsequently, part of the cells cultures were transdifferentiated according to the conditions detailed in Table 3.

TABLE 2
Seeding conditions

| | | Well | | Medium | |
|---|---|---|---|---|---|
| | Cell | size | Cell density | volume | |
| | number | (cm²) | (cells/cm²) | (ml) | Cells/ml |
| Standard | 100,000 | 9.5 | ~10,000 | 4 | 25,000 |
| 6 wells plates | | | | | |
| Ultra low | 300,000 | 9.5 | ~30,000 | 4 | 75,000 |
| attachment | | | | | |
| 6 wells plates | | | | | |
| 75T flask | 2,500,000 | 75 | ~33,000 | 25 | 100,000 |

TABLE 3
Transfection and cell culture conditions

| | | | | Cell | Plate |
|---|---|---|---|---|---|
| Group | Treatment | Media | Conditions | number | type |
| 1 | Untreated | TD + | adherent | 100,000 | 6 well plate* |
| 2 | TD | serum | adherent | 100,000 | 6 well plate * |
| 5 | Untreated | | non-adherent | 2,500,000 | 75T flask |
| 6 | TD | SFM + | non-adherent | 2,500,000 | 75T flask |
| 7 | Untreated | ITS | non-adherent | 300,000 | 3X wells** |
| 8 | TD | | non-adherent | 300,000 | 3X wells** |

Harvesting on day 6: cell cultures grown in 75T Flasks were collected and transfered to 50 ml conical tubes, centrifuged at 500 RPM for 5 min, and 15 ml of medium was removed. Cells (aprox 2.5×10⁶ in 10 ml medium) were divided and used for the assays detailed in Table 4. Cells grown in ultra-low attachment 6 wells plates were transferred to adherent 6 wells plates for GSIS analysis. Transfer to non-adherent conditions was done in 4 ml SFM collected from the flasks. No fresh medium was added to imitate the conditions of clusters cultured in 6 wells plates.

TABLE 4
Assays performed on cells grown in 75T flasks

| | | Medium | | |
|---|---|---|---|---|
| | | volume | Cells | |
| | Assay | (ml) | seeded | Repeats |
| 1 | GSIS in cell inserts | 1.5 | 375,000 | 2 |
| 2 | RNA | 1 | 250,000 | 2 |
| 3 | Transfer to 2D | 1 | 250,000 | 3 |
| 4 | Cell dissociation with | 0.75 | 187,500 | 1 |
| | Accutase | | | |
| 6 | Trypsinization with | 0.75 | 187,500 | 1 |
| | trypsin | | | |

Harvesting on day 7: 3D cell clusters were collected and cell number was counted. A portion of cells from the clusters were re-seeded in non-adherent conditions for additional GSIS assay, wherein other portions were used for RNA extraction.

Morphology of Cell Spheroids

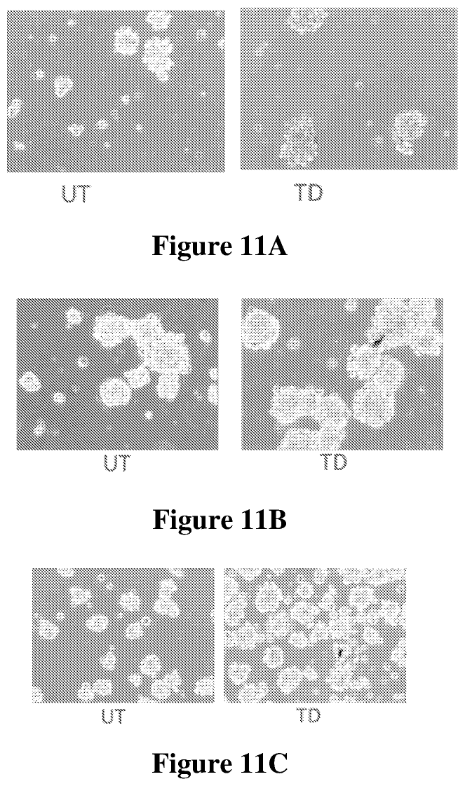

Both non-transdifferentiated and transdifferentiated cells grown in ultra-low attachment 75T flasks formed smaller clusters than cells grown in 6 wells plates. Clusters in 75T flasks were uniformly distributed thorough the flask area (FIG. 11A). Cells clusters observed in non-adherent 6 wells plates were similar to 3D clusters observed in previous experiments. 3D clusters formed in 6 well plates were larger, they were positioned in the center of the well and attached to each other to some extent (FIG. 11B). After being transferred to adherent 6 wells plates, 3D cell clusters originated in 75T flasks positioned in the center of the well and then adhered to the its surface (FIG. 11C).

3D Cell Clusters Maintain their Phenotype when Re-Seeded under Adherent Conditions

To determine optimal conditions for transdifferentiation, the expression levels of ectopically expressed PDX-1, NeuroD1 and MafA, as well as of pancreatic β cell gene markers GCG and NKX6.1, were measured in transdifferentiated and untreated cells cultured under adherent and non-adherent conditions in 6 well plates and under non-adherent conditions in 75T flasks.

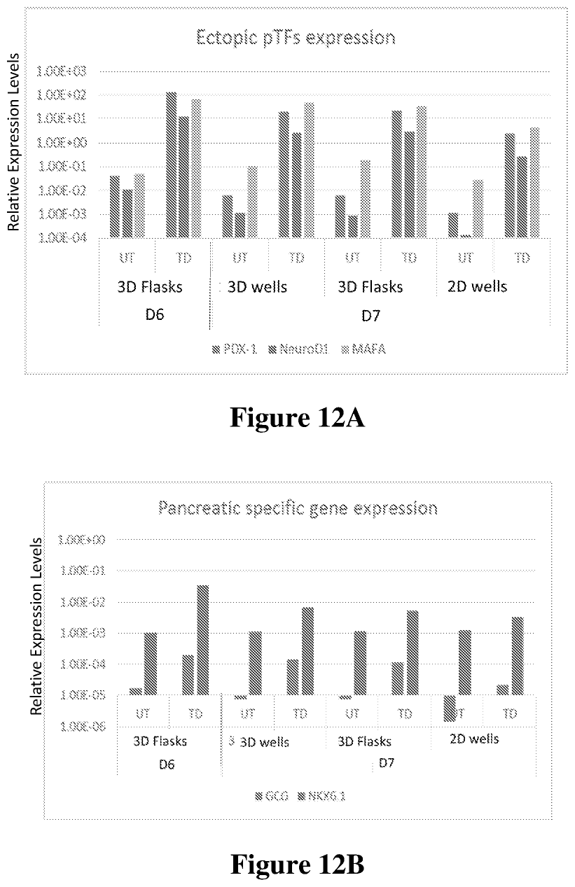

Non-adherent culture conditions were found to increase the expression of ectopically expressed PDX-1, NeuroD1 and MafA, as well as of GCG and NKX6.1, compared to adherent culture conditions (FIG. 12A and FIG. 12B). Cells re-seeded under adherent conditions and previously grown in 75T flasks or in ultra-low attachment 6 wells plates showed similar expression of ectopically expressed PDX-1, NeuroD1 and MafA, and of GCG and NKX6.1 (FIG. 12A and FIG. 12B).

Cells harvested at day 6 showed higher expression of ectopically expressed PDX-1, NeuroD1 and MafA compared to cells harvested on day 7 (FIG. 12A). Unexpectedly, the expression of GCG and NKX6.1 was also higher in cells harvested on day 6 (FIG. 12B), suggesting that an additional culturing day, re-seeding under adherent conditions, or the additional GSIS assay negatively affected the maturation of the cells towards a pancreatic β cells phenotype.

Levels of NKX6.1 expression in the positive control used in this assay (human islet) was 4 times lower than usual. This may explain the low levels of NKX6.1 detected in the current experiment.

A summary of the RT-PCR results is presented in Table 5, showing threshold cycle numbers for all analyzed groups.

TABLE 5
RT-PCR threshold cycle numbers presented in FIG. 12A and 12B.

| | Culture | | | | | | | |
|---|---|---|---|---|---|---|---|---|
| Day | conditions | Treatment | PDX1 | NeuroD1 | MafA | GCG | NKX6.1 | SST |
| D6 | 3D-flask | UT | 4.21E−02 | 1.11E−02 | 4.95E−02 | 1.72E−05 | 1.02E−03 | 3.37E−04 |
| | | TD | 1.3E+02 | 12.6 | 66.7 | 1.92E−04 | 3.36E−02 | 3.37E−04 |
| D7 | 3D-well | UT | 6.4E−03 | 1.13E−02 | 1.01E−01 | 7.42E−06 | 1.11E−03 | 1.34E−04 |
| | | TD | 20.08 | 46.9 | 46.9 | 1.39E−04 | 6.79E−03 | 1.21E−04 |
| | 3D-flask | UT | 6.34E−03 | 1.82E−01 | 1.82E−01 | 7.41E−06 | 1.17E−03 | |
| | | TD | 21.75 | 33.5 | 33.5 | 1.14E−04 | 5.08E−03 | |
| | 2D | UT | 1.15E−03 | 2.76E−02 | 2.76E−02 | 1.42E−04 | 1.22E−03 | 3.64E−05 |
| | | TD | 2.35 | 2.78E−01 | 4.22 | 2.11E−05 | 3.37E−03 | 3.94E−05 |

Accutase® Effectively Detaches Cells from 3D Cell Clusters

The efficiency of Accutase® and trypsin for dissociating cells from 3D clusters was compared in cell clusters generated in 75T flasks. Accutase desintegrated clusters efficiently. While survival rates of dissociated cells was still low when using Accutase, with survival rates of 71% for untreated and 78% for transdifferentiated cells, it was a great improvement compared with trypsinization. Trypsin detached only a few individual cells from 3D clusters, and those detached cells were dead.

### Example 5: Viruses from Different Manufacturers

Objective: To validate previous results by using adenoviruses provided by other manufacturers.

Study design and methods: Primary adult liver human cells were obtained from 2 different donors and transdifferentiated according to the methods described above. Cells were transdifferentiated as described in Example 1. Primary liver cells were either infected with adenoviruses provided by OD260 Inc. (ID, USA) or by Pall Inc. (USA). Following infection, cells were seeded either in adherent 6 wells microplates or in ultra-low attachment T75 Flasks in CMRL-SFM medium. Untreated control cells were cultured in similar conditions. On day 6 cells were harvested and RNA was extracted.

### Culture of Transdifferentiated Cells in Non-Adherent Conditions Promote Mature Pancreatic β Cell Phenotype

Expression levels of pancreatic β cell gene markers, as well as of ectopically expressed PDX-1, NeuroD1 and MafA, were measured in transdifferentiated cells cultured under adherent and non-adherent conditions in 6 well plates, and infected with Pall Inc. (USA) adenoviruses. Non transdifferentiated cells were used as control.

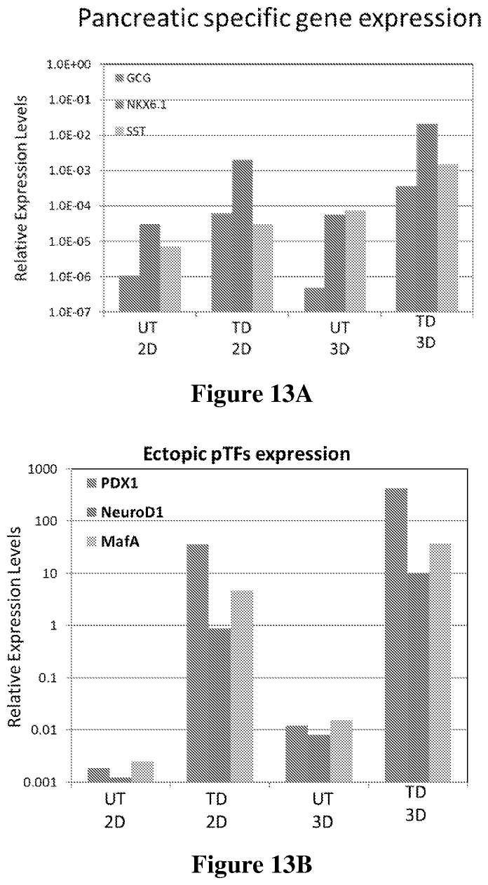

Non-adherent culture conditions were found to increase the expression of GCG, NKX6.1 and SST (FIG. 13A), as well as of ectopically expressed PDX-1, NeuroD1 and MafA (FIG. 13B), compared to adherent culture conditions.

Similar Transdifferentiation Efficacy is Obtained by Infecting Cells with Viruses from Different Manufacturers

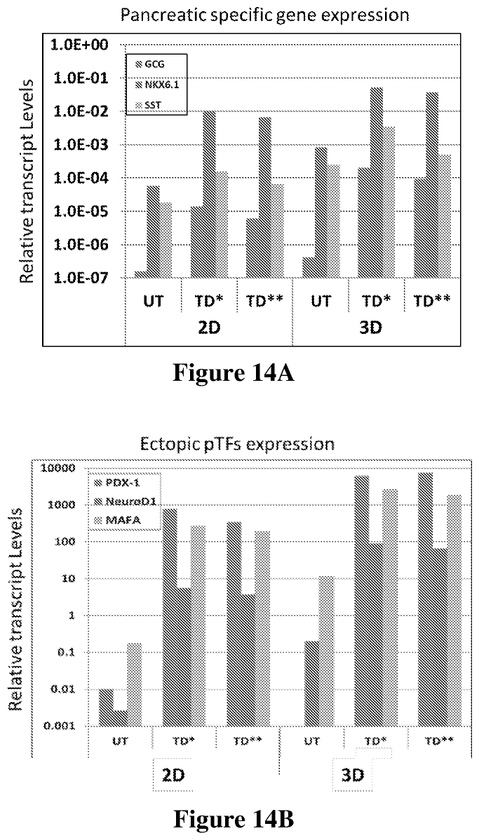

Expression levels of pancreatic β cell gene markers, as well as of ectopically expressed PDX-1, NeuroD1 and MafA, were measured in cells transdifferentiated with similar viruses manufactured either by OD260 Inc. (ID, USA) or Pall Inc. (USA). No significant differences were found between the groups (FIG. 14A and FIG. 14B, *: cells infected with OD260 Inc. (ID, USA) adenoviruses; **: cells infected with Pall Inc. (USA) adenoviruses).

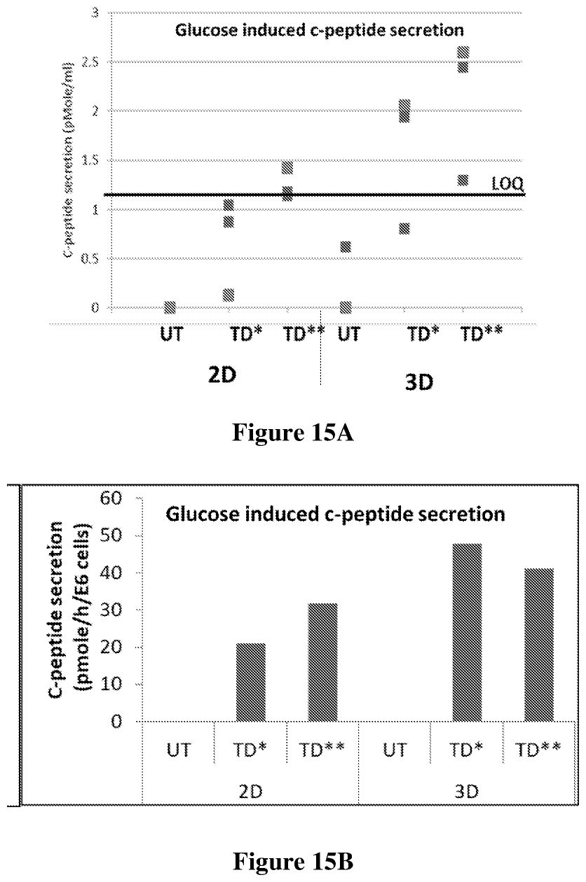

Glucose regulated C-peptide secretion was compared between cells cultured under adherent and non-adherent conditions, and transfected with OD260 or Pall viruses. Cells grown in non-adherent conditions showed improved glucose regulated C-peptide secretion compared to cells grown on adherent conditions. Cell transfected with OD260 and Pall viruses showed similar levels of C-peptide secretion (FIGS. 15A and 15B, *: cells infected with OD260 Inc. (ID, USA) adenoviruses; **: cells infected with Pall Inc. (USA) adenoviruses).

### Example 6: Generation of 3D Clusters in Microwells

Objective: To develop methods for generating three-dimensional (3D) cell clusters of a predetermined size.

Study design and methods: Cells were transdifferentiated as described in Example 1. Following MafA infection on day 3, cells were seeded in AggreWell™ 400 plates (StemCell Technologies). AggreWell™ 400 plates were used to generate cell aggregates in a reproducible and highly uniform manner. Each well of the AggreWell™ plate contains a standardized array of microwells of 400 μm³. The size of the clusters can be controlled by adjusting the input cell density. Prior to use and following the manufacturer's instructions, each well was rinsed with an Anti-Adherence Rinsing Solution that prevents cell adhesion and promotes efficient aggregate formation. Then, single-cell suspensions of untreated (UT) and transdifferentiated (TD) cells were prepared in SFM medium. Cells were counted to determine the viable cell concentration, and 150 cells were seeded in each well. The cells were evenly distributed in the well and centrifuged at 100×g for 3 minutes to capture cells in the microwells. AggreWell plates were incubated at 37° C. with 5% CO2 and 95% humidity for 2 weeks, and were evaluated for aggregate formation under a light microscope. Cells were harvested at days 7 or 15 for RNA extraction for RT-PCR studies.

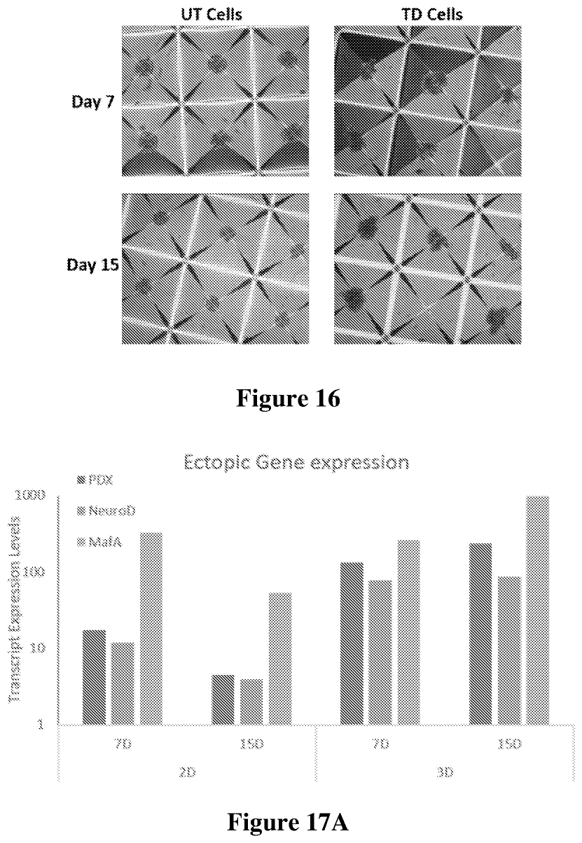

Results: TD cells formed larger clusters compared to UT cells both at days 7 and 15. Additionally, TD cell clusters showed higher coefficient of variance (CV) percentages than UT cells. Table 6 summarizes the observed cluster sizes and CVs of TD and UT cells. FIG. 16 shows representative 3D cell clusters of TD and UT cells at days 7 and 15.

TABLE 6
Size of 3D cell clusters produced in Aggrewell ™ plates.

| | | Day 15 - | | Day 7- |
|---|---|---|---|---|
| | Day 15 - | Average | Day 7 - | Average |
| | CV | Cluster size | CV | Cluster size |
| Treatment | (%) | (μm) | (%) | (μm) |
| TD (150 | 16% | 156 | 11% | 140 |
| cells/microwell) | | | | |
| UT (150 | 9% | 117 | 5% | 113 |
| cells/microwelb | | | | |

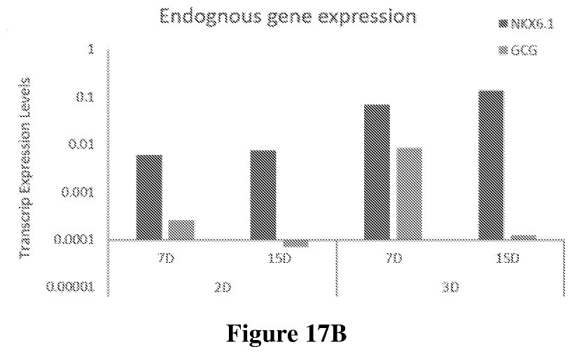

Gene expression profiles were evaluated on TD cells that were grown either as monolayers (2D) or in AggreWell to form aggregates (3D). Gene expression was measured on days 7 and 15. The expression of ectopically expressed Pdx-1, NeuroD1 and MafA, and of endogenous pancreatic genes Nkx6.1 and GCG was measured. Higher ectopic gene expressions was seen in TD cells grown as 3D clusters compared to TD cells grown in 2D, both at days 7 and 15 (FIG. 17A). Higher Nkx6.1 and GCG gene expression levels were observed in TD cells grown as 3D clusters compared to TD cells grown in 2D, both at days 7 and 15 (FIG. 17B).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (cancelled)

14. (canceled)

15. (cancelled)

16. (canceled)

17. A method of generating a three-dimensional (3D) cell cluster comprising transdifferentiated mammalian non-pancreatic beta cells having a mature pancreatic beta cell phenotype and a scaffold; wherein at least a subset of said cells are attached to said scaffold, the method comprising:
(a) providing a scaffold;
(b) obtaining primary adult mammalian non-pancreatic cells;
(c) propagating and expanding the cells of step (b) to a predetermined number of cells;
(d) transdifferentiating the cells of step (c); wherein said transdifferentiating comprises:
(i) infecting said expanded cells with an adenoviral vector comprising a nucleic acid encoding a human PDX-1 polypeptide;
(ii) infecting said expanded cells of step (i) with an adenoviral vector comprising a nucleic acid encoding a second human pancreatic transcription factor polypeptide; and
(iii) infecting said expanded cells of step (ii) with an adenoviral vector comprising a nucleic acid encoding a human MafA polypeptide;
and a step of attaching at least a subset of said cells to said scaffold after step (b), (c), or (d);
thereby generating a 3D cell cluster comprising transdifferentiated mammalian non-pancreatic beta insulin producing cells, wherein at least a subset of said cells are attached to said scaffold.

18. (canceled)

19. The method of claim 1, wherein said second pancreatic transcription factor is selected from NeuroD1 and Pax4.

20. The method of claim 1, wherein steps (i) and (ii) are concurrent.

21. The method of claim 1, wherein step (c), step (d), or a combination thereof are executed under non-adherent cell culture conditions.

22. The method of claim 1, wherein said scaffold is selected from a group comprising: a solid scaffold, a hydrogel, an extracellular matrix, an extracellular matrix hydrogel, a protein hydrogel, a peptide hydrogel, a polymer hydrogel, a wood-based nanocellulose hydrogel, polyglycerol sebacate (PGS), or any combination thereof.

23. (canceled)

24. (canceled)

25. (canceled)

26. (canceled)

27. (canceled)

28. The method of claim 1, wherein said 3D cell cluster is encapsulated by an encapsulation agent.

29. The method of claim 1, wherein said encapsulation agent comprises a material selected from a group comprising: alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, and low molecular weight dextran sulphate (LMW-DS), or any derivatives thereof, and any combination thereof.

30. (canceled)

31. The method of claim 1, wherein said transdifferentiated cells comprise
(a) improved glucose regulated C-peptide secretion or insulin secretion; increased
(b) GCG, NKX6.1, or PAX6 expression; increased expression of the ectopic pancreatic transcription factors used for transdifferentiation; or increased insulin content; compared to transdifferentiated non-pancreatic beta insulin producing cells cultured as a monolayer cell culture; or
secretion of at least 20 pmole/h*10⁶ cells of C-peptide in response to high glucose concentrations; or
(c) any combination thereof.

32. (canceled)

33. (canceled)

34. The method of claim 1, wherein said adult mammalian non-pancreatic beta cells are selected from the group comprising epithelial cells, endothelial cells, keratinocytes, fibroblasts, muscle cells, hepatocytes, liver cells, blood cells, stem or progenitor cells, liver stem cells, neural stem cells, mesenchymal stem cells, hematopoietic stem; progenitor cells, bone marrow stem cells, umbilical cord blood stem cells, peripheral blood stem cells, fetal liver stem cells, or adipose tissue stem cells, or any combination thereof.

35. (canceled)

36. A method for treating a pancreatic disease or disorder in a subject, the method comprising administering a 3D cell cluster comprising transdifferentiated mammalian non-pancreatic beta cells having a mature pancreatic beta cell phenotype and a scaffold to said subject; wherein at least a subset of said cells are attached to said scaffold, and wherein said 3D cell cluster is produced by a method comprising:
(a) providing a scaffold;
(b) obtaining primary adult mammalian non-pancreatic cells;
(c) propagating and expanding the cells of step (b) to a predetermined number of cells:
(d) transdifferentiating the cells of step (c) wherein said transdifferentiating comprises:
(i) infecting said expanded cells with an adenoviral vector comprising a nucleic acid encoding a human PDX-1 polypeptide;
(ii) infecting said expanded cells of step (i) with an adenoviral vector comprising a nucleic acid encoding a second human pancreatic transcription factor polypeptide; and
(iii) infecting said expanded cells of step (ii) with an adenoviral vector comprising a nucleic acid encoding a human MafA polypeptide;
and a step of attaching at least a subset of said cells to said scaffold after step (b), (c), or (d);
thereby treating said disease in said subject.

37. The method of claim 36, wherein said administering comprises intradermal, intraperitoneal, or surgical administration, or any combination thereof, of said 3D cell cluster to said subject.

38. The method of claim 37, wherein said disease comprises type I diabetes, type II diabetes, gestational diabetes, pancreatic cancer, hyperglycemia, pancreatitis, pancreatic pseudocysts, pancreatic trauma caused by injury, type 3 diabetes or a complication of pancreatectomy, or any combination thereof.

39. A three-dimensional (3D) cell cluster comprising transdifferentiated adult mammalian non-pancreatic beta cells having a mature pancreatic beta cell phenotype and function and a scaffold, wherein at least a subset of said cells are attached to said scaffold, and wherein said cells are transdifferentiated by a method comprising
(a) obtaining primary adult mammalian non-pancreatic cells;
(b) propagating and expanding the cells of step (b) to a predetermined number of cells;
(c) infecting said expanded cells with an adenoviral vector comprising a nucleic acid encoding a human PDX-1 polypeptide;
(d) infecting the cells of step (c) with an adenoviral vector comprising a nucleic acid encoding a second human pancreatic transcription factor polypeptide; and
(e) infecting the expanded cells of step (d) with an adenoviral vector comprising a nucleic acid encoding a human MafA polypeptide.
wherein said transdifferentiated cells increased insulin secretion; compared to transdifferentiated non-pancreatic beta insulin producing cells cultured as a monolayer cell culture.

40. The 3D cell cluster of claim 39, wherein said scaffold is selected from a group comprising: a solid scaffold, a hydrogel, an extracellular matrix, an extracellular matrix hydrogel, a protein hydrogel, a peptide hydrogel, a polymer hydrogel, a wood-based nanocellulose hydrogel, polyglycerol sebacate (PGS), or any combination thereof.

41. The 3D cell cluster of claim 39, wherein said 3D cell cluster is encapsulated by an encapsulation agent.

42. The 3D cell cluster of claim 41, wherein said encapsulation agent comprises a material selected from a group comprising: alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, and low molecular weight dextran sulphate (LMW-DS), or any derivatives thereof, and any combination thereof.

43. The 3D cell cluster of claim 39, wherein said transdifferentiated cells comprise
(a) improved glucose-regulated C-peptide or insulin secretion; increased GCG NKX6.1, or PAX6 expression; increased expression of the ectopically expressed transcription factors; or increased insulin content; compared to transdifferentiated non-pancreatic beta insulin producing cells cultured as a monolayer cell culture; or
(b) secretion of at least 20 pm/h*10⁶ cells of C-peptide in response to high glucose concentrations;
(c) any combination thereof.

44. The 3D cell cluster of claim 39, wherein said adult mammalian non-pancreatic beta cells are selected from the group comprising epithelial cells, endothelial cells, keratinocytes, fibroblasts, muscle cells, hepatocytes, liver cells, blood cells, stem or progenitor cells, liver stem cells, neural stem cells, mesenchymal stem cells, hematopoietic stem or progenitor cells, bone marrow stem cells, umbilical cord blood stem cells, peripheral blood stem cells, fetal liver stem cells, or adipose tissue stem cells, or any combination thereof.

45. A pharmaceutical composition comprising the 3D cell cluster of claim 39.
